SEQUENCE LISTING (5 sequences)

SEQ ID NO: 1 - DNA, 3054 bp, Saccharomyces cerevisiae
  source 1..3054: mol_type = genomic DNA
atgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggaaagccttaccattcaaatgccaaaggtgatttggcacaaattttacagcttaccagatattttgctgggtccgctgataagtttgacaaaggtgcaaccataccattgacttttaacaagtttgcatatactctaaaagttccttttggcgttgttgctcaaatcgttccatggaattatcctctagctatggcttgttggaaattgcaaggtgccttagcagccggtaacacggttatcatcaaacctgctgagaatacctctctatctctactttattttgctactttaattaaaaaagcaggttttccacctggtgttgtcaatatcgttcctggttatggatcacttgtaggccaagccctagcatctcacatggatatcgacaaaatatcttttacgggaagcaccaaggtcggtggatttgtgttggaagcttccggccaatcgaaccttaaagacgttacactagaatgcggtggtaagtctcctgctctcgtatttgaagatgcagaccttgataaggctatcgattggatagcagctggcattttctacaattcaggacagaattgtaccgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaagggaggaaaagttggacatgttccagacctctgaatttcctattggtggagctaaaggctacttcattcccccaaccatcttcactgatgtcccgcaaacatcgaaactgttacaggatgagatatttggcccggttgtggttgttagcaagttcacaaattatgatgacgctctgaagctggctaatgatacttgctacgggctcgcctctgcggtcttcacaaaagatgtcaagaaagcgcacatgtttgctcgcgatattaaagcaggaactgtttggatcaactcatctaacgatgaagatgttaccgttccttttggcgggtttaaaatgagtggtattggtagagaactggggcaaagtggtgttgatacctatcttcaaacaaaagcaagatatagattatacacatttagaaaattagccaaaagaaaaatgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggtaagcctttccattccaatgctaaacaagacttagcccagattatagaacttacaagatactatgcgggggcggtcgacaagttcaatatgggtgaaaccattccattgacttttaacaagtttgcatatactctaaaagttccttttggcgttgttgctcaaatcgttccatggaattatcctctagctatggcttgtagaaaaatgcaaggtgccttagcggccggtaacacggttatcatcaaacctgctgaaaatacctctctatctctactttattttgctactttaattaaaaaagcaggttttccacctggtgttgtcaatgtcattcctggttatggttccgttgtggggaaagctttaggaacccacatggatatcgacaaaatatcttttacgggaagtactaaggttggcggctcagtattggaagcttccggccaatcgaaccttaaggatatcacactagaatgcggtggtaagtctcctgctcttgtatttgaagatgcagaccttgataaggctatagaatgggtagcaaatggtattttttttaattcgggacagatctgcactgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaaaggaggaaaagttggacatgttccagacctctgaatttcctattggtggagctaaaggctacttcattcccccaaccatcttcactgatgtaccagaaacatctaagttgctgcgtgatgaaatatttggcccggttgtggttgttagcaagttcacaaattatgatgacgctctgaagctggctaatgatacttgctacgggctcgcctctgcggtcttcaccaaagatgtcaagaaagcgcacatgtttgctcgcgatattaaagcaggaactgtttggatcaatcaaaccaatcaagaagaagctaaagttccttttggcggatttaagatgagtggtattggtagagaatcaggcgacaccggcgttgataactatttacaaataaaatcagtccatgtggatctttcattggataaataa

SEQ ID NO: 2 - DNA, 42 bp, Saccharomyces cerevisiae
  source 1..42: mol_type = genomic DNA
agatatagattatacacatttagaaaattagccaaaagaaaa

SEQ ID NO: 3 - DNA, 1521 bp, Saccharomyces cerevisiae
  source 1..1521: mol_type = genomic DNA
atgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggaaagccttaccattcaaatgccaaaggtgatttggcacaaattttacagcttaccagatattttgctgggtccgctgataagtttgacaaaggtgcaaccataccattgacttttaacaagtttgcatatactctaaaagttccttttggcgttgttgctcaaatcgttccatggaattatcctctagctatggcttgttggaaattgcaaggtgccttagcagccggtaacacggttatcatcaaacctgctgagaatacctctctatctctactttattttgctactttaattaaaaaagcaggttttccacctggtgttgtcaatatcgttcctggttatggatcacttgtaggccaagccctagcatctcacatggatatcgacaaaatatcttttacgggaagcaccaaggtcggtggatttgtgttggaagcttccggccaatcgaaccttaaagacgttacactagaatgcggtggtaagtctcctgctctcgtatttgaagatgcagaccttgataaggctatcgattggatagcagctggcattttctacaattcaggacagaattgtaccgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaagggaggaaaagttggacatgttccagacctctgaatttcctattggtggagctaaaggctacttcattcccccaaccatcttcactgatgtcccgcaaacatcgaaactgttacaggatgagatatttggcccggttgtggttgttagcaagttcacaaattatgatgacgctctgaagctggctaatgatacttgctacgggctcgcctctgcggtcttcacaaaagatgtcaagaaagcgcacatgtttgctcgcgatattaaagcaggaactgtttggatcaactcatctaacgatgaagatgttaccgttccttttggcgggtttaaaatgagtggtattggtagagaactggggcaaagtggtgttgatacctatcttcaaacaaaagcagttcacataaatctctctttggacaactaa

SEQ ID NO: 4 - DNA, 1521 bp, Saccharomyces cerevisiae
  source 1..1521: mol_type = genomic DNA
atgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggtaagcctttccattccaatgctaaacaagacttagcccagattatagaacttacaagatactatgcgggggcggtcgacaagttcaatatgggtgaaaccattccattgacttttaacaagtttgcatatactctaaaagttccttttggcgttgttgctcaaatcgttccatggaattatcctctagctatggcttgtagaaaaatgcaaggtgccttagcggccggtaacacggttatcatcaaacctgctgaaaatacctctctatctctactttattttgctactttaattaaaaaagcaggttttccacctggtgttgtcaatgtcattcctggttatggttccgttgtggggaaagctttaggaacccacatggatatcgacaaaatatcttttacgggaagtactaaggttggcggctcagtattggaagcttccggccaatcgaaccttaaggatatcacactagaatgcggtggtaagtctcctgctcttgtatttgaagatgcagaccttgataaggctatagaatgggtagcaaatggtattttttttaattcgggacagatctgcactgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaaaggaggaaaagttggacatgttccagacctctgaatttcctattggtggagctaaaggctacttcattcccccaaccatcttcactgatgtaccagaaacatctaagttgctgcgtgatgaaatatttggcccggttgtggttgttagcaagttcacaaattatgatgacgctctgaagctggctaatgatacttgctacgggctcgcctctgcggtcttcaccaaagatgtcaagaaagcgcacatgtttgctcgcgatattaaagcaggaactgtttggatcaatcaaaccaatcaagaagaagctaaagttccttttggcggatttaagatgagtggtattggtagagaatcaggcgacaccggcgttgataactatttacaaataaaatcagtccatgtggatctttcattggataaataa

SEQ ID NO: 5 - DNA, 829 bp, Saccharomyces cerevisiae
  source 1..829: mol_type = genomic DNA
ggatctttccgtagggtgaacctggcggagagggatcattaaagaaatttaataattttgaaaatggatttttttgttttggcaagagcatgagagcttttactgggcaagaagacaagagatggagagtccagccgggcctgcgcttaagtgcgcggtcttgctaggcttgtaagtttctttcttgctattccaaacggtgagagatttctgtgcttttgttataggacaattaaaaccgtttcaatacaacacactgtggagttttcatatctttgcaactttttctttgggcattcgagcaatcggggcccagaggtaacaaacacaaacaattttatctattcattaaatttttgtcaaaaacaagaattttcgtaactggaaattttaaaatattaaaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttccttctcaaacattctgtttggtagtgagtgatactctttggagttaacttgaaattgctggccttttcattggatgttttttttccaaagagaggtttctctgcgtgcttgaggtataatgcaagtacggtcgttttaggttttaccaactgcggctaatctttttttatactgagcgtattggaacgttatcgataagaagagagcgtctaggcgaacaatgttcttaaagtttgacctcaaatccaggtaggagtccccaacgccc

(19) United States
(12) Patent Application Publication
(10) Pub. No.: US 2024/0189400 A1
(43) Pub. Date: Jun. 13, 2024

(54) FOOD AND DRUG COMPOSITION CONTAINING NOVEL ALDEHYDE DEHYDROGENASE FOR SUPPRESSING TREMOR OR MOVEMENT DISORDER

(71) Applicant: PICOENTECH Co., LTD., Seongnam-Si, Gyeonggi-Do (KR)

(72) Inventor: Hung Taeck KWON, Seoul (KR)

(73) Assignee: PICOENTECH Co., LTD., Seongnam-Si, Gyeonggi-Do (KR)

(21) Appl. No.: 18/519,569

(22) Filed: Nov. 27, 2023

(30) Foreign Application Priority Data
Dec. 1, 2022 (KR) 10-2022-0165794
Nov. 7, 2023 (KR) 10-2023-0153075

(51) Int. Cl. A61K 38/44; A61K 9/00; A61K 36/064

(52) U.S. Cl. CPC A61K 38/44; A61K 9/0056; A61K 36/064; C12Y 102/01003

(57) ABSTRACT

A food and drug composition for suppressing tremor or movement disorder, containing novel aldehyde dehydrogenase encoded by a gene having more than 98% homology to the gene of SEQ ID NO: 1. In addition, the embodiments relate to a food composition and pharmaceutical composition for suppressing tremor or movement disorder, containing a lysate of any one or a mixture thereof selected from KCTC13925BP, KCTC 14122BP, KCTC14123BP, KCTC14983BP, KCTC14984BP, and KCTC14985BP.

### REFERENCE TO A SEQUENCE LISTING

In accordance with 37 CFR § 1.831, the present specification makes reference to a Sequence Listing submitted electronically as an .xml file named “PKPA2301KRPR1USA.xml”. The .xml file was generated on Jan. 12, 2024, and is 13000 bytes in size. The entire contents of the Sequence Listing are hereby incorporated by reference.

### TECHNICAL FIELD

The present invention relates to a food and drug composition for suppressing tremor or movement disorder, containing novel aldehyde dehydrogenase encoded by a gene having more than 98% homology to the gene of SEQ ID NO: 1. Specifically, the present invention relates to a food and drug composition, containing an aldehyde dehydrogenase encoded by the gene of SEQ ID NO: 1 including SEQ ID NO: 2.

In addition, the present invention relates to a food composition and pharmaceutical composition for suppressing tremor or movement disorder, containing a lysate of any one or a mixture thereof selected from the group consisting of KCTC13925BP, KCTC 14122BP, KCTC14123BP, KCTC14983BP, KCTC14984BP, and KCTC14985BP.

### Background

Tremor or movement disorder (hereinafter abbreviated as tremor) is a symptom that occurs not only in the hands but also in the head, neck, jaw, tongue, and voice. Although these tremor symptoms are rarely diagnosed, they can occur throughout the body, including the legs and feet.

Tremor symptoms are classified into essential tremor, resting tremor, parkinsonian tremor, psychogenic tremor, cerebellar tremor, intention tremor, alcohol withdrawal tremor and orthostatic tremor.

In the case of essential tremor, the cause has not yet been identified. More than 50% of patients with essential tremor have a family history. Essential tremor occurs at all ages. Essential tremor that occurs at the age of 65 or older is referred to as senile tremor.

Resting tremor and Parkinsonian tremor occur in Parkinson's disease patients. This tremor mainly occurs when the patient is resting, and decreases or disappears while the patient performs the desired action.

Factitious or psychogenic tremor is also called hysterical tremor. In this type of tremor, when the patient's attention is diverted from the area where the tremor is occurring, the tremor may disappear.

When abnormal movements continue or when detailed movement control is required, irregular tremors may appear temporarily. This is called cerebellar tremor or intention tremor. This type of tremor symptom is most often caused by abnormalities in the cerebellum or its connection to the cerebellum. Patients with cerebellar dysfunction exhibit abnormal movement symptoms such as ataxia and difficulty controlling movements.

Tremor that is observed with the naked eye when the amplitude of the tremor increases for some reason, such as anxiety, nervousness, stage fright, or an important competition, is called enhanced physiologic tremor.

In addition to these tremor symptoms, there are alcohol withdrawal tremor and orthostatic tremor.

Additionally, acetaldehyde, which causes hangovers caused by excessive drinking, also causes symptoms of tremor or movement disorder. Malondialdehyde, which rapidly increases in the bodies of athletes during intense sports such as soccer or basketball, causes symptoms such as tremors or movement disorders such as painful muscle spasms.

In general, hand tremor refers to a case where tremor symptoms such as hand tremor appear. These hand tremors include resting tremor, postural tremor, kinetic tremor, task-specific tremor, and intention tremor.

Hand tremors are one of the most common movement disorders caused by functional abnormalities in the nerves. Hand tremors occur in approximately 1% of the world's population. The incidence of tremor increases with age.

The main symptom of tremor is activity tremor, which is tremor that occurs during voluntary activity. This type of tremor gradually develops as aging progresses, ultimately causing difficulties in daily activities such as eating and driving, resulting in problems that impair the quality of life.

In addition to basic neurological examination, several methods are used to examine patients with tremors and objectively evaluate their symptoms. For academic purposes, the Fahn-Tolosa-Marin scale (FTM) method is widely used. An assessment of daily living disorders is also conducted.

Movement tremor can be objectively evaluated by having the patient draw an Archimedes spiral or observing the handwriting and the way the patient writes. Tremor symptoms can be objectively measured using acceleration measurement. In most patients, detailed visual observation of tremor symptoms is important for diagnosis.

The meaning of the word “tremor” in this specification includes all the various tremor or movement disorder symptoms listed above.

In the treatment of essential tremor (tremor), medications are used to improve symptoms. The most widely used drugs are propranolol and primidone. Propranolol is a non-selective beta-adrenergic antagonist, and primidone is a drug developed as an antiepileptic drug. They have an inhibitory effect on essential tremor.

When primidone is used in combination with propranolol, it shows greater therapeutic effect than monotherapy. Clonazepam is a tremor treatment drug suitable for patients with diabetes and asthma, or patients who have difficulty taking primidone.

Essential tremor is a common symptom and can be improved with treatment. However, many patients do not receive treatment for essential tremor because they perceive it as a phenomenon caused by age. Because essential tremor is caused by a wide variety of causes, the cause of tremor must be accurately diagnosed.

The exact cause of neurodegenerative diseases such as Alzheimer's disease (AD) and Parkinson's disease (PD), including essential tremor, has not yet been accurately identified. According to recent studies, it is believed that the main factor in neurodegenerative diseases is increased oxidative stress in nerve cells.

Oxidative stress is caused by the intracellular accumulation of reactive aldehyde. These reactive aldehydes include endogenous aldehydes such as Glutamate semialdehyde (GSA), Succinic semialdehyde (SSA) derived from glutamine, or acetaldehyde generated from ethanol.

These reactive aldehydes increase oxidative stress and cause inflammatory reactions by combining with major proteins, DNA, and lipids in the body. Oxidative stress and chronic inflammation in the human body accelerates aging of the human body. As people age, neurodegenerative diseases that cause tremors or abnormal movements may occur.

There are about 19 types of ALDH (aldehyde dehydrogenase) in the human body that can decompose these endogenous aldehydes. Among these ALDHs, mitochondrial ALDH2 plays an important role in the endogenous aldehyde degradation process.

ALDH is responsible for removing these endogenous aldehydes and protects cells from oxidative stress. ALDH stabilizes cells by acting as a free radical scavenger and prevents the accumulation of active aldehydes in the body. As a result, ALDH protects against neuronal death and suppresses the occurrence of tremors or abnormal movements due to neurodegeneration.

Succinic semialdehyde (SSA), which is derived from endogenous acetaldehyde, malondialdehyde, and γ-aminobutyric acid (GABA), which functions as a neurotransmitter, has been pointed out as the cause of tremor symptoms. These substances are the cause of the diseases that the food composition and pharmaceutical composition of the present invention are intended to prevent or treat.

GABA is produced in the body from its precursor, glutamate. It is converted into GABA aldehyde (succinic semialdehyde) by the action of MAOB (monoamine oxidase B). GABA aldehyde (succinic semialdehyde, hereinafter abbreviated as SSA) is oxidized to succinic acid by the action of ALDH in the human body.

Meanwhile, the tremor symptom that appears during the onset of spastic paraplegia is related to glutamate semialdehyde (GSA, Glutamate-5-semialdehyde), a metabolite of glutamate.

When the expression of human aldehyde dehydrogenase (ALDH) is reduced or mutated, the decomposition of acetaldehyde, malondialdehyde, glutamate semialdehyde (GSA), and succinic semialdehyde (SSA) in the body is delayed. As a result, oxidative stress in the human body increases and causes motor nerve abnormalities, causing symptoms of tremors or movement disorders.

### Summary

Through various previous studies as described above, it has been found that as acetaldehyde, malondialdehyde, glutamate semialdehyde (GSA), or succinic semialdehyde (SSA) increases in the body, symptoms of tremor or movement disorder occur.

Therefore, the primary purpose of the present invention is to provide a food composition and a pharmaceutical composition containing aldehyde dehydrogenase which rapidly converts glutamate semialdehyde (GSA), an endogenous aldehyde derived from acetaldehyde, malondialdehyde, and glutamate, and succinic semialdehyde (SSA) into an acid compound.

That is, the purpose of the present invention is to provide a food composition or pharmaceutical composition that suppresses various tremor symptoms that occur due to abnormalities in the oxidation and excretion process of acetaldehyde, malondialdehyde, succinic semialdehyde (SSA), and glutamate semialdehyde (GSA) due to incomplete functioning of aldehyde dehydrogenase (ALDH) in the human body.

Another object of the present invention is to provide a food composition or pharmaceutical composition for suppressing tremor, containing aldehyde dehydrogenase (ALDH) encoded by the gene of SEQ ID NO: 1 including SEQ ID NO: 2.

Still yet another object of the present invention is to provide a food composition or pharmaceutical composition for suppressing tremor, which comprise dried powder (hereinafter abbreviated as KARC) of a lysate of any one or a mixture thereof selected from the group consisting of KCTC13925BP, KCTC14122BP, KCTC14123BP, KCTC14983BP, KCTC14984BP, and KCTC14985BP.

The primary object of the present invention as described above can be achieved by providing a food composition and a pharmaceutical composition containing aldehyde dehydrogenase contained in lysate of any one or a mixture thereof selected from the group consisting of KCTC13925BP, KCTC14122BP, KCTC14123BP, KCTC14983BP, KCTC14984BP, and KCTC14985BP.

The object of the present invention as described above can be achieved by providing a food composition and a pharmaceutical composition that can converts acetaldehyde, Succinic semialdehyde (SSA), and Glutamate semialdehyde (GSA) into acid compounds, comprising aldehyde dehydrogenase contained in lysate of any one or a mixture thereof selected from the group consisting of KCTC13925BP, KCTC14122BP, KCTC14123BP, KCTC14983BP, KCTC14984BP, and KCTC14985BP.

The compositions of the present invention containing KARC, shows the effect for promoting the dehydrogenation reaction of Succinic semialdehyde (SSA), Glutamate-5-semialdehyde (GSA), acetaldehyde, or malondialdehyde which are suspected to be the cause of tremor symptoms or abnormal movement symptoms including hand tremors.

### BRIEF DESCRIPTION OF DRAWINGS

FIG. 1 is a chemical formula showing the production and decomposition process of endogenous aldehydes in vivo.

FIG. 2 is a chemical formula showing the production and decomposition process of endogenous aldehydes in vivo.

Ethanol or ethanol derivatives (2-substitued ethanol, R—CH2CH2-OH) are in vivo reversibly converted to acetaldehyde derivatives (R—CH2-CHO) by alcohol dehydrogenase (ADH). Acetaldehyde derivatives, highly toxic substances are irreversibly converted to relatively non-toxic acetic acid derivatives (R—CH2-CO2H).

Endogenous monoamine (R—CH2CH2-NH2) is converted to aldehyde (R—CH2-CHO) by monoamine oxidase (MAO) enzyme, followed by aldehyde dehydrogenase and alcohol dehydrogenase reactions detoxified to acetic acid (R—CH2-CO2H). It's the same way as alcohol metabolism.

FIG. 3 shows the decomposition of GABA in vivo.

The monoamine, neurotransmitter GABA is oxidized by the monoamine oxidase (MAO) enzyme and converted into endogenous aldehydes, Succinic semialdehyde (SSA), thereby binding and denaturing surrounding proteins. In result, the accumulation of denatured proteins within the endoplasmic reticulum acts as a cytotoxic agent to induce cell death. [FIG. 3].

FIG. 4 is a graph showing the ability of KARC of the present invention to decompose endogenous acetaldehyde. These results show that the composition of the present invention decomposes endogenous acetaldehyde and suppresses self-brewing symptoms.

FIG. 5 is a graph showing the malondialdehyde decomposition ability of KARC.

In animal experiments in which blood malondialdehyde [FIG. 5] which are endogenous toxic aldehydes, were increased by consuming alcohol,

FIG. 7 is a graph showing the ability of KARC of the present invention to decompose acetaldehyde in the human body.

FIG. 8 is a graph showing the ability of KARC to decompose malondialdehyde in the human body.

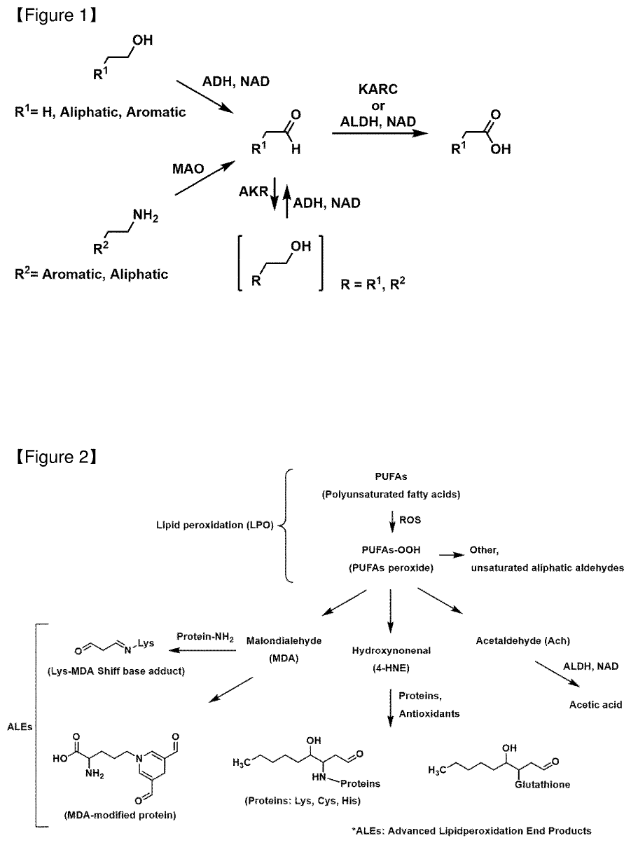

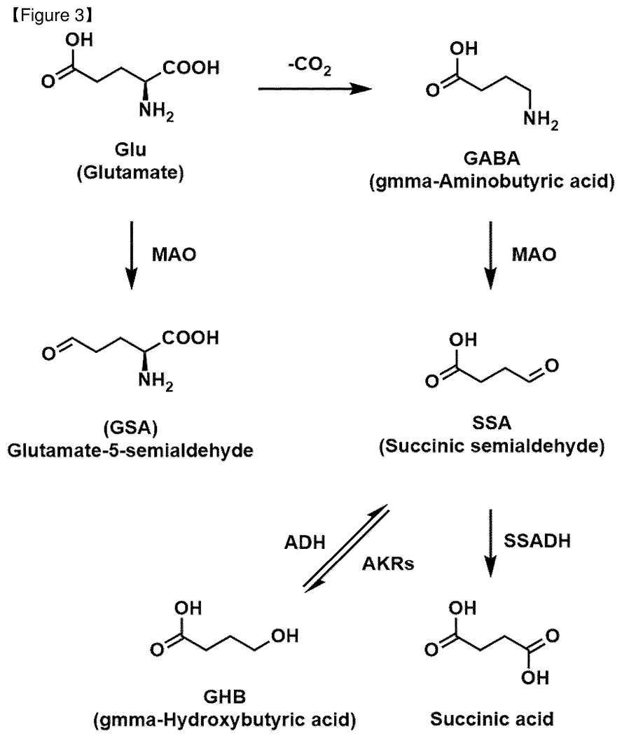

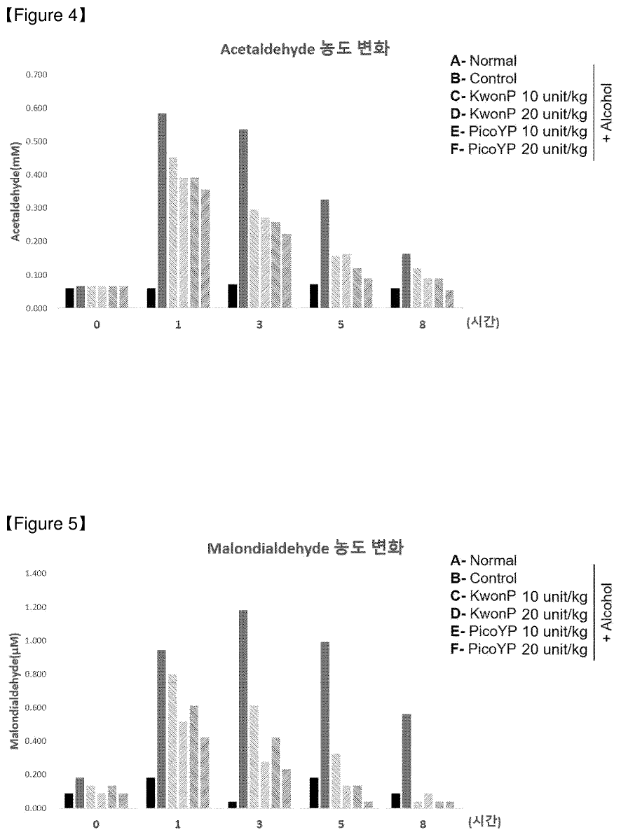

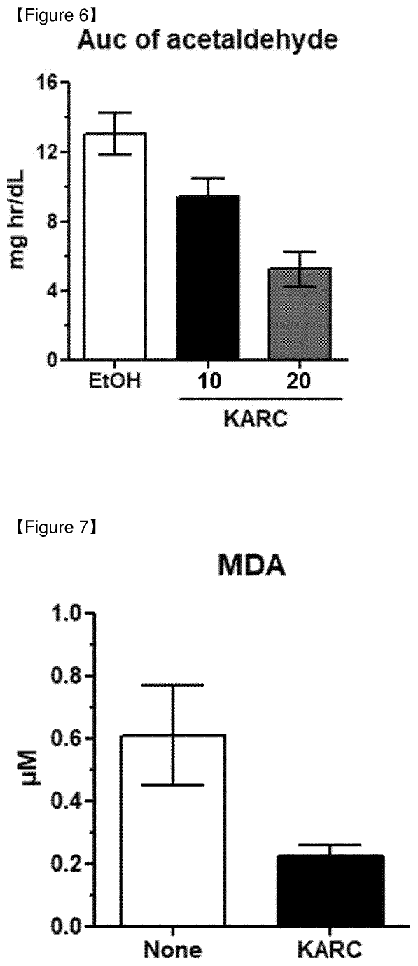

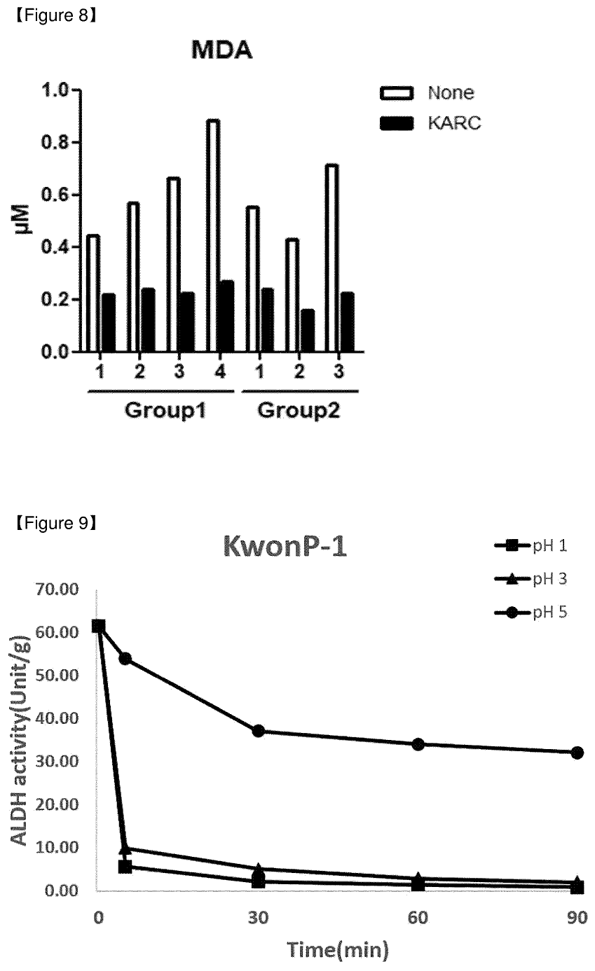

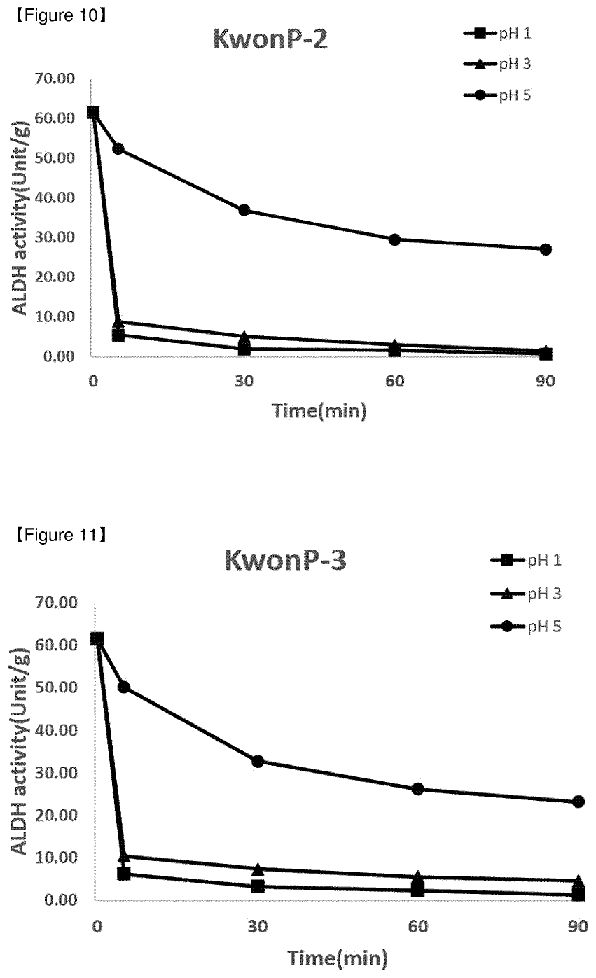

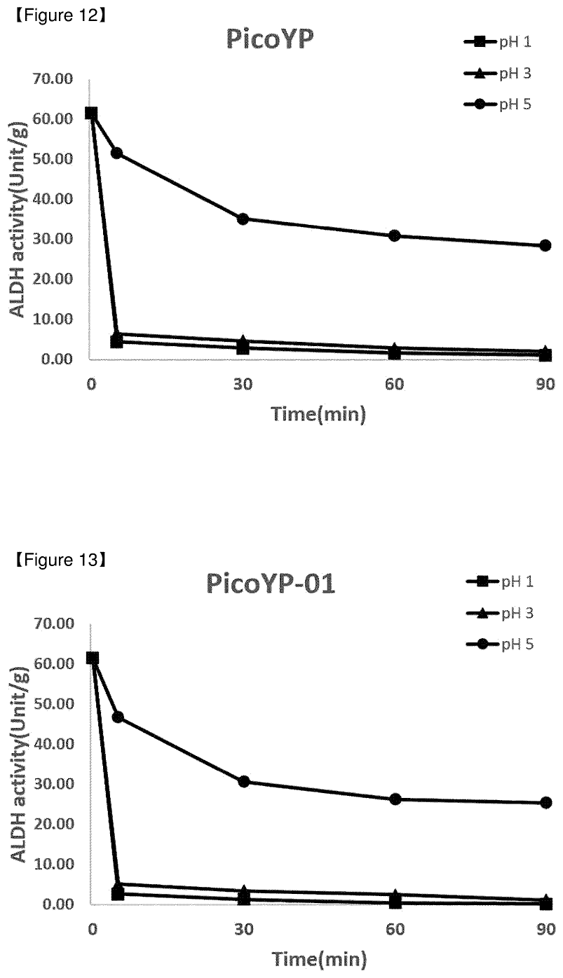

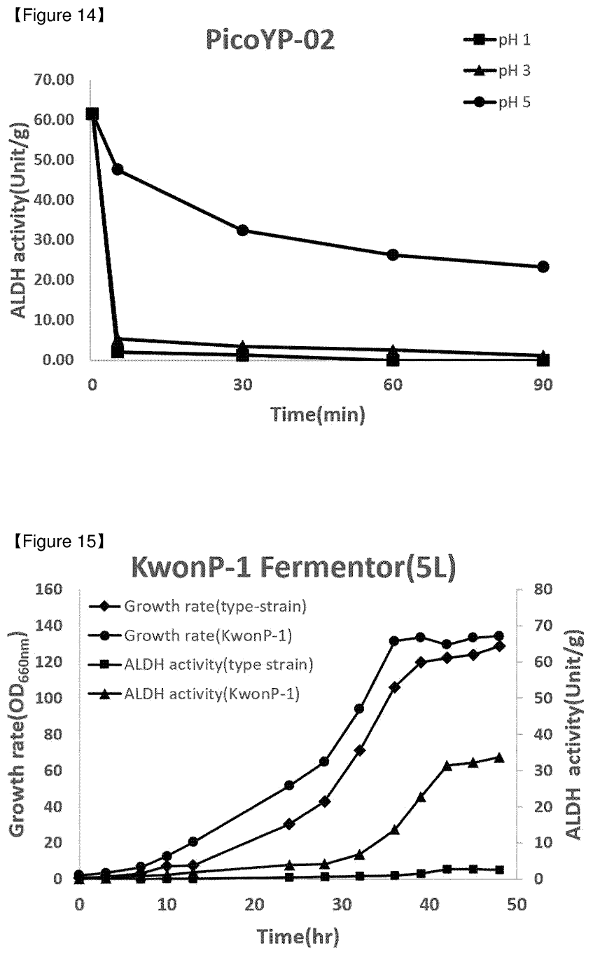

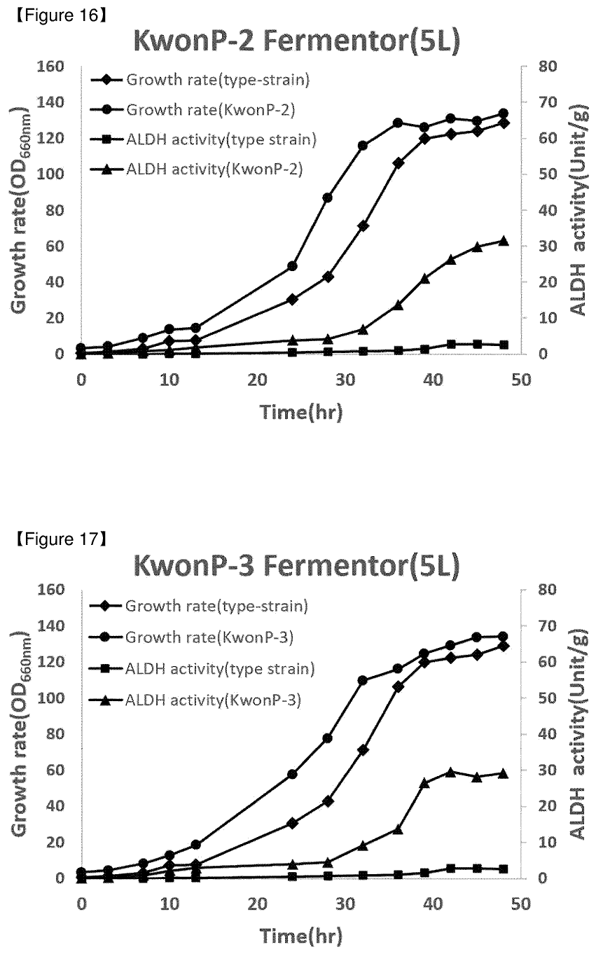

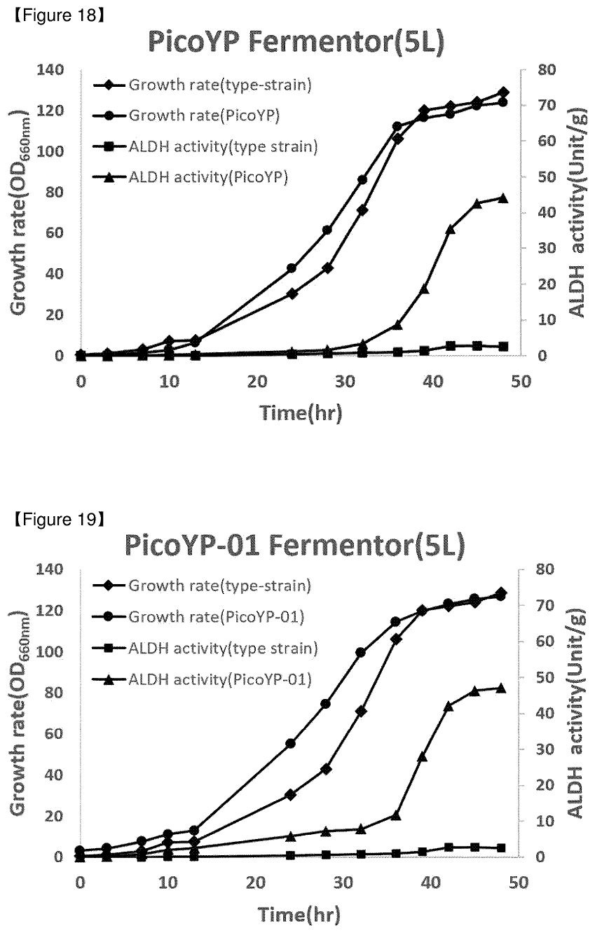

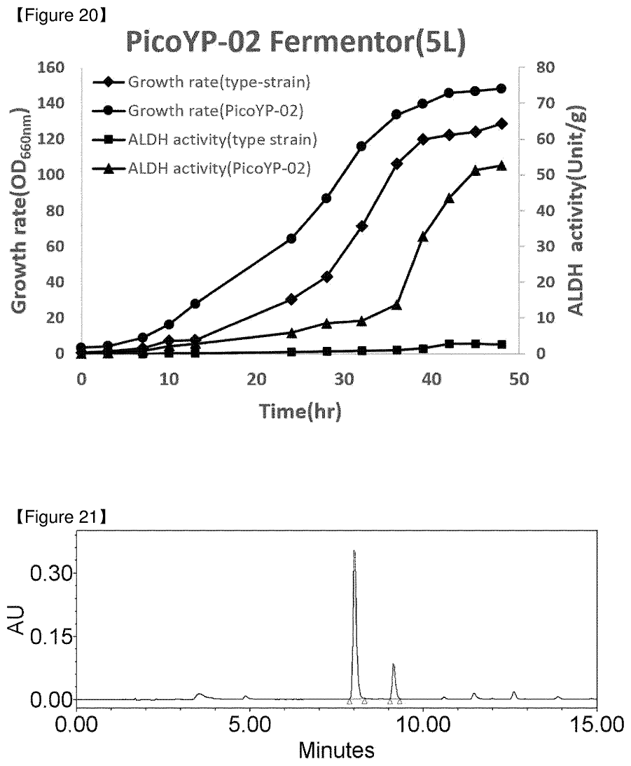

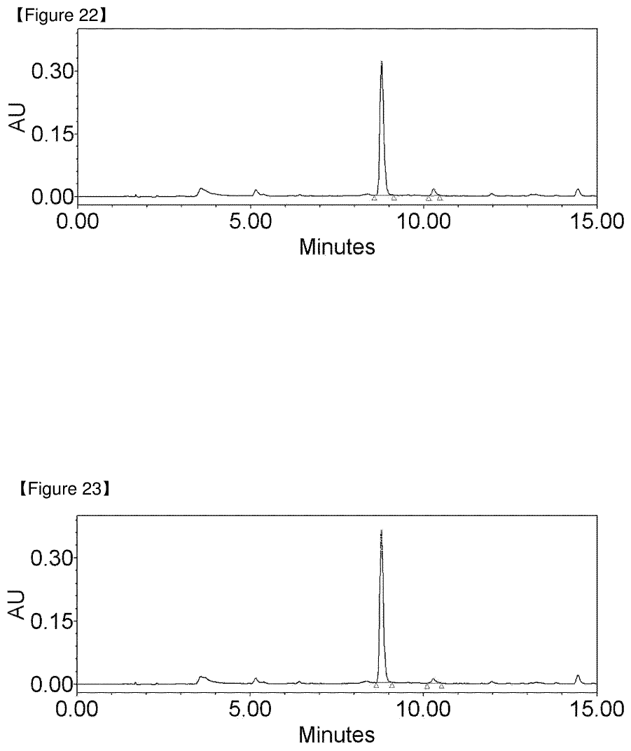

In tests, the reduction of endogenous blood acetaldehyde and malondialdehyde were confirmed in animal [FIGS. 4, 5, 6] and in human [FIGS. 7, 8], This shows that tremor symptoms, including hand tremors, or movement disorders can be suppressed by lowering oxidative stress caused by drinking and fatigue. [FIGS. 4, 5, 6, 7].

In [FIG. 8], in a state where oxidative stress was increased due to taking medicine, etc. and after taking KARC, malondialdehyde, a biomarker of oxidative stress and active oxygen, was lowered. It was confirmed the effect of KARC alleviating Tremor symptom by oxidative stress.

FIG. 9 shows changes in enzyme activity when the KwonP-1 strain included in KARC of the present invention is orally administered.

FIG. 10 shows changes in enzyme activity when the KwonP-2 strain included in KARC of the present invention is orally administered.

FIG. 11 shows changes in enzyme activity when the KwonP-3 strain included in KARC of the present invention is orally administered.

FIG. 12 shows changes in enzyme activity when the PicoYP strain included in KARC of the present invention is orally administered.

FIG. 13 shows changes in enzyme activity when the PicoYP-01 strain included in KARC of the present invention is orally administered.

FIG. 14 shows the change in enzyme activity when the PicoYP-02 strain included in KARC of the present invention is orally administered.

[FIGS. 9, 10, 11, 12, 13, 14] shows KwonP-1, KwonP-2, KwonP-3, Pico YP, Pico YP-01, and PicoYP-02 were orally administered under conditions (1<pH<5) similar to the digestive process of the stomach in human for 90 minutes. The change in ALDH enzyme activity were measured.

ALDH enzyme activity was maintained at a minimum of 37.29 unit/g and a maximum of 52.24% at pH=5 (similar to condition observed during food intake). It was confirmed that the enzyme activity was maintained when KARC was administered orally.

FIG. 15 shows the growth curve and enzyme activity of KwonP-1 strain cultured in a 5 L fermenter.

FIG. 16 shows the growth curve and enzyme activity of KwonP-2 strain cultured in a 5 L fermenter.

FIG. 17 shows the growth curve and enzyme activity of KwonP-3 strain cultured in a 5 L fermenter.

FIG. 18 shows the growth curve and enzyme activity of Pico YP strain cultured in a 5 L fermenter.

FIG. 19 shows the growth curve and enzyme activity of PicoYP-01 strain cultured in a 5 L fermenter.

FIG. 20 shows the growth curve and enzyme activity of PicoYP-02 strain cultured in a 5 L fermenter.

In [FIGS. 15, 16, 17, 18, 19, 20], the novel mutant strains: KwonP-1, KwonP-2, KwonP-3, PicoYP, PicoYP-01, and PicoYP-01 were respectively cultured using YPD medium in a 5 L fermenter under the same conditions. It was carried out at 30° C. and 200 rpm for 48 hours.

When comparing the growth curve (OD660 nm) and ALDH enzyme activity of each strain with that of the type strain, ALDH enzyme activity was at least 10.5 times and up to 18.75 times higher. PicoYP-01 had the highest ALDH activity at 52.68 unit/g, and KwonP-3 had the lowest at 29.5 unit/g.

FIG. 21 is the HPLC spectrum of a mixture of distilled water and SSA.

FIG. 22 is an HPLC spectrum after maintaining the mixture of KARC and SSA of the present invention at 37° C. for 1 hour.

FIG. 23 is an HPLC spectrum after maintaining the mixture of KARC and SSA of the present invention at 37° C. for 3 hours.

In [FIGS. 21, 22, 23], when KARC was treated at 37° C., there was a 55% decrease in 1 hour and a 74.9% in 3 hours. KARC oxidized SSA, metabolite of GABA.

### DETAILED DESCRIPTION OF THE EMBODIMENTS

Hereinafter, the method for producing dry powder of KARC of the present invention, the lysate of Saccharomyces cerevisiae KCTC13925BP, KCTC14122BP, KCTC14123BP KCTC14983BP, KCTC14984BP, KCTC14985BP, will be described in more detail.

However, these examples are for illustrative purposes only of compositions that can achieve the purpose of the present invention, and therefore, the scope of the present invention is not limited only to the compositions described in the following examples.

### [Example 1] Screening Wild Yeast Parent Strain to Proceed Mutation

In the present invention, each suspension of traditional Korean wines (hereinafter referred to as makgeolli) was prepared by mixing various types of makgeolli with a 0.9% NaCl solution. The makgeolli suspension was stirred at 200 rpm for 1 hour. The supernatant containing the yeast wild strain was diluted with YPD yeast extract peptone dextrose broth) medium. The diluted solution was prepared to be 10⁻⁶ times the original solution.

The diluted solution was smeared on YPD agar medium. The agar medium was statically cultured at 30° C. under aerobic conditions for one week. Saccharomyces cerevisiae was primary screened based on morphological characteristics of colonies, growth characteristics at YM medium and microscopic observation.

The ALDH activity and glutathione content of screened Saccharomyces cerevisiae were measured. Parent strain was selected based on ALDH activity and glutathione production.

### 1-1: Measurement of Aldehyde Dehydrogenase

Acetaldehyde reacted with Dinitrophenylhydrazine (DNPH) to form acetaldehyde-hydrazone (Ach-DNPH) compound. Ach-DNPH compounds were detected at 360 nm by HPLC equipped with a C18 column. The amount of aldehyde reduced by the decomposition reaction by aldehyde dehydrogenase ALDH) was quantified through the amount of the detected Ach-DNPH compound.

The enzyme reaction was carried out at 30° C. by adding 10ul of the yeast lysate to 990ul reaction mixture [50 mM potassium phosphate buffer (pH 8.0), 1.5 mM acetaldehyde and 3 mM NADP+]. After the enzyme reaction was completed, 50 ul of 10 mM DNPH was added to induce the formation of Ach-DNPH. Ach-DNPH formation proceeded at 22° C. for 1 hour.

Ach-DNPH formation was terminated by addition of 3M sodium acetate (pH 9). The Ach-DNPH compound formed was separated by adding twice volume of acetonitrile. The separated Ach-DNPH compound (in ACN) was analyzed by injection into HPLC.

The concentration of the Ach-DNPH compound was analyzed at a wavelength of 360 nm by setting HPLC under the condition of developing a mobile phase (acetonitrile, water) on a C18 column at a rate of 1 ml/min. The area value of the chromatogram obtained as a result of HPLC was converted using the material standard curve of aldehyde-DNPH (Sigma-Aldrich) to quantify the concentration of the Ach-DNPH compound. The reduced concentration of Ach-DNPH per minute, 1 mM, was calculated as 1 unit of ALDH. The activity of ALDH was standardized as Unit/mg-protein.

### 1-2: Glutathione Measurement

Yeast cells were harvested by centrifuging 1 ml of Saccharomyces cerevisiae culture medium. A suspension was prepared by adding 1 ml of water to the harvested yeast cells. Glutathione was extracted by stirring the suspension at 1,000 rpm at 85° C. for 2 hours. The suspension was centrifuged to remove yeast cells, and the supernatant was filtered through a 0.22 μm filter to obtain a sample containing glutathione.

The concentration of glutathione in the sample was analyzed by HPLC (Shimazu LC-20AD) equipped with a C18 column. The concentration of glutathione was analyzed at a wavelength of 210 nm under conditions in which the mobile phase (2.02 g/L Sodium 1-heptanesulfonate monohydrate, 6.8 g/L Potassium dihydrogen phosphate, pH 3.0, methanol mixture) was developed at a rate of 1 ml/min. The area value of the chromatogram obtained as a result of HPLC was analyzed using the standard curve of glutathione.

ALDH activity and glutathione content were analyzed for 200 different types of yeast obtained from Korean makgeolli. The 10 types of yeast listed in [Table 1] had higher ALDH activity or glutathione production ability than other yeasts.

The ALDH activity of Yeast #97 was 0.10 Unit/mg-protein, the second highest overall. The glutathione content of Yeast #97 was 0.42%, the highest among all of yeast 97 was selected as the parent strain and a mutation induction procedure was performed.

TABLE 1

| | ALDH activity | Glutathione | |
|---|---|---|---|
| strain | (Unit/mg-protein) | content (%) | Screening |
| Yeast #6 | 0.06 | 0.38 | |
| Yeast #18 | 0.11 | 0.14 | |
| Yeast #22 | 0.08 | 0.38 | |
| Yeast #41 | 0.14 | 0.22 | |
| Yeast #97 | 0.10 | 0.42 | Selected parent strain |
| | | | (Wild-Type) |
| Yeast #109 | 0.10 | 0.36 | |
| Yeast #112 | 0.09 | 0.40 | |
| Yeast #126 | 0.10 | 0.28 | |
| Yeast #168 | 0.11 | 0.38 | |
| Yeast #197 | 0.08 | 0.41 | |

### [Example 2] Identification of the Parent Strain Used in the Mutagenesis Process

Identification was performed to confirm the exact species of the wild-type parent strain (Yeast #97, Wild-type yeast). To ensure sufficient yeast cells for DNA extraction, only colonies of a single yeast were plated on YPD agar medium. DNA was extracted using a Genomic DNA prep kit (HiGene™, BIOFACT Co., Ltd., Daejeon, Korea) according to the manufacturer's instructions

To amplify rRNA gene on ITS region of the yeast, polymerase chain reaction (PCR) was performed on yeast chromosomal DNA using the ITS5 (forward) and ITS4 (reverse) primers. DNA sequencing of PCR result was analyzed.

The DNA sequence of the parent strain was isolated using the Bioedit program. The reverse strand of the PCR result was converted into a paired base sequence through a reverse completion process.

It was confirmed that the sequence of the forward strand matched the paired sequence of the reverse strand by the Cluster X program. The parent strain which was matching the sequence information confirmed through the above experimental process was identified by using the BLAST database provided by the U.S. National Center for Biotechnology Information (NCBI). As a result of identification, it was found that rRNA in the ITS of the parent strain was 100% identical to that of Saccharomyces cerevisiae.

### [Example 3] Selection of Mutant Strains with Improved Aldehyde Dehydrogenase Production

The mutation induction process for the wild-type Saccharomyces cerevisiae parent strain was conducted according to the method described in U.S. patent application Ser. No. 17/176,365.

To induce mutations in the yeast parent strain, wild yeast strains that produce both ALDH and glutathione were treated with ethyl methane sulfonate (EMS) or nitrosoguanidine (NGD). Yeast strains in which mutations were induced were exposed to various concentrations of methylglyoxal. A mutant strain with excellent adaptability to methylglyoxal was selected. Selected yeast strains were exposed to various concentrations of lysine. A mutant strain with excellent adaptability to lysine was selected. Thirty mutant strains with excellent adaptability to methylglyoxal and lysine were obtained. Each of the 30 yeasts was evaluated through five characteristics: growth curve, ALDH activity, ADH activity, coenzyme content, and glutathione content.

### 3-1: Growth Characteristics

Saccharomyces cerevisiae is a crab tree positive microorganism and produces ethanol simultaneously with growth under aerobic conditions. Cultivating yeast with high yields requires Saccharomyces cerevisiae with high ethanol tolerance.

YPD media with different ethanol concentrations (no ethanol, 5%, 7%, and 10%) were prepared. Culture medium of Saccharomyces cerevisiae(yeast) adjusted to OD=1 at 660 nm was prepared. Each mixture of the prepared YPD medium and yeast culture medium was diluted at a ratio of 99:1. Finally, YPD media containing yeast with four different concentrations of alcohol were prepared. Each YPD medium mixed with yeast was cultured with shaking at 30° C. and 200 rpm. The growth curve of the mutant strain was measured every 3 hours for 48 hours. The growth curve of each mutant strains is evaluated through three characteristics: time (or period) of lag phase, specific growth rate (OD660 nm/hr) of exponential phase, and maximum density (OD660 nm).

The higher concentration of ethanol in YPD medium, the longer the time taken for the lag phase. The maximum density and specific growth rate decreased. As a result of comparing the maximum density of mutant strains at low concentration (ethanol 5%) and high concentration (ethanol 10%), it was found that in the case of nine mutant strains, 50% of growth was even maintained at high concentration compared to growth at low concentration. The growth characteristics of the nine mutant strains that distinguished them from other strains were a short lag phase and a high specific growth rate.

TABLE 2

| # | 5% ethanol | | | 7% ethanol | | | 10% ethanol | | | |
|---|---|---|---|---|---|---|---|---|---|---|
| # | hr | OD_{660 nm}/hr | OD_{660 nm} | hr | OD_{660 nm}/hr | OD_{660 nm} | hr | OD_{660 nm}/hr | OD_{660 nm} | Selection |
| 1 | 9 | 0.6477 | 22.2 | 15 | 0.5210 | 16.5 | 24 | 0.1968 | 4.81 | |
| 2 | 12 | 0.3675 | 12.34 | 24 | 0.1835 | 4.11 | 36 | 0.0140 | 0.212 | |
| 3 | 9 | 0.4285 | 15.8 | 15 | 0.2888 | 9.12 | 24 | 0.0880 | 2.16 | |
| 4 | 15 | 0.9683 | 25.3 | 15 | 0.8815 | 25.3 | 15 | 0.4085 | 12.4 | K-1 |
| 5 | 12 | 0.7368 | 14.12 | 15 | 0.7337 | 12.23 | 21 | 0.2205 | 5.68 | |
| 6 | 12 | 0.2590 | 9 | 15 | 0.1773 | 5.44 | 33 | 0.0353 | 0.448 | |
| 7 | 24 | 0.9664 | 22.3 | 27 | 0.8467 | 16.8 | 30 | 0.2673 | 5.17 | |
| 8 | 6 | 0.7268 | 23 | 9 | 0.6222 | 21.5 | 15 | 0.3778 | 12.11 | K-2 |
| 9 | 6 | 0.8433 | 22.12 | 9 | 0.7484 | 25.34 | 15 | 0.3005 | 9.41 | |
| 10 | 3 | 0.4880 | 14.5 | 18 | 0.2013 | 6.12 | 24 | 0.0808 | 2.14 | |
| 11 | 3 | 0.2766 | 11.15 | 9 | 0.2223 | 8.22 | 24 | 0.0988 | 2.41 | |
| 12 | 6 | 0.7149 | 21.68 | 9 | 0.5969 | 20.52 | 12 | 0.3317 | 11.4 | K-3 |
| 13 | 9 | 0.6106 | 22.4 | 12 | 0.4906 | 16.4 | 15 | 0.1813 | 5.68 | |
| 14 | 12 | 0.6136 | 20.6 | 24 | 0.3060 | 6.85 | 36 | 0.0278 | 0.41 | |
| 15 | 12 | 0.4759 | 15.45 | 18 | 0.1751 | 5.41 | 33 | 0.0707 | 0.896 | |
| 16 | 9 | 0.8533 | 23.8 | 15 | 0.8065 | 20.9 | 21 | 0.4953 | 12.1 | K-4 |
| 17 | 21 | 0.6016 | 14.85 | 24 | 0.3955 | 8.45 | 24 | 0.0547 | 1.26 | |
| 18 | 12 | 0.7766 | 19.25 | 18 | 0.4437 | 12.4 | 24 | 0.1219 | 2.85 | |
| 19 | 3 | 0.5050 | 14.75 | 9 | 0.4463 | 14.6 | 21 | 0.2521 | 6.23 | |
| 20 | 27 | 0.0666 | 1.41 | 36 | 0.0278 | 0.36 | — | — | — | |
| 21 | 3 | 0.6044 | 22.14 | 9 | 0.6051 | 20.64 | 12 | 0.3247 | 11.1 | K-5 |
| 22 | 24 | 0.5798 | 13.4 | 27 | 0.5080 | 10.1 | 30 | 0.1604 | 3.1 | |
| 23 | 15 | 0.6455 | 16.9 | 15 | 0.5877 | 16.9 | 15 | 0.2003 | 6.3 | |
| 24 | 3 | 0.7269 | 20.4 | 6 | 0.6375 | 18.6 | 12 | 0.3522 | 10.5 | K-6 |
| 25 | 9 | 0.4858 | 16.7 | 15 | 0.3908 | 12.4 | 24 | 0.1476 | 3.6 | |
| 26 | 6 | 0.6559 | 17.2 | 9 | 0.5821 | 19.7 | 15 | 0.3177 | 9.6 | K-7 |
| 27 | 9 | 0.2857 | 10.5 | 15 | 0.1925 | 6.1 | 24 | 0.0587 | 1.4 | |
| 28 | 12 | 0.6315 | 12.1 | 15 | 0.6289 | 10.5 | 21 | 0.1890 | 4.9 | |
| 29 | 6 | 0.5451 | 17.3 | 9 | 0.4667 | 16.1 | 15 | 0.2834 | 9.1 | K-8 |
| 30 | 9 | 0.7826 | 21.8 | 9 | 0.6614 | 19.9 | 15 | 0.3267 | 10.6 | K-9 |

### 3-2: Activity of Alcohol Dehydrogenase ADH) and Aldehyde Dehydrogenase ALDH)

The activity of alcohol dehydrogenase (ADH) was measured by adding 10 μl of yeast lysate to 990 μl of the reaction mixture with the composition of 50 mM potassium phosphate buffer (pH 8.0), 2 mM NAD+ and 1% ethanol. The activity of aldehyde dehydrogenase (ALDH) was measured by adding 10 μl of yeast lysate to 990 μl of the reaction mixture with the composition of 50 mM potassium phosphate buffer (pH 8.0), 3 mM NAD+ and 1.5 mM acetaldehyde. The enzymatic reaction of ADH and ALDH was carried out at 30° C. for 5 minutes, and the concentration of NAD(P)H produced as a result of the enzyme reaction was measured through absorbance at 340 nm.

The enzyme activities of nine mutant strains (K-1 to K-9) selected in the present invention were measured. The ADH activity of the mutant strain was a minimum of 382.69 units/g and a maximum of 975.29 units/g. The ADH activity of the mutant strain increased at least 5.1 times and up to 13.1 times compared to the type strain (reference yeast, Saccharomyces cerevisiae KCTC7296). The ALDH activity of the mutant strain was a minimum of 15.23 unit/g and a maximum of 72.16 unit/g. The ALDH activity of the mutant strain increased by at least 5.3 and up to 24.9 times compared to the enzyme activity of the type-strain.

Six mutant strains (K-1, 4, 6, 7, 8, and 9) showed similar increase rate of enzyme activity of ADH and ALDH compared to the type strain. The enzyme activity of ALDH in the three mutant strains (K-2, 3, and 5) was 18.3, 23.2 and 24.9 times higher, respectively, compared to the type-strain. The enzyme activity of ADH in the three mutant strains (K-2, 3, and 5) was 9.7, 11.6, and 13.1 times higher respectively, compared to the type-strain. The rate of increase in enzyme activity of ALDH for the three mutant strains (K-2, 3, and 5) was twice as high as that of ADH.

The present inventors named three novel mutant strains (K-2, 3, and 5) adapted to increase aldehyde dehydrogenase (ALDH) activity as PicoYP, PicoYP-01, and PicoYP-02, respectively. The three novel mutant strains were deposited at the Korea Research Institute of Bioscience and Biotechnology's Biological Resources Center and were assigned the deposit numbers of KCTC14983BP, KCTC14984BP, and KCTC14985BP, respectively.

### 3-3: Content of Coenzyme (NAD and NADP)

NADtotal and NADPtotal in lysates extracted from mutant strains were measured with NADH/NAD+ assay kit and NADPH/NADP+ assay kit, respectively. NAD(P) in the sample was converted to NAD(P)H using NAD(P) cycling buffer and NAD(P) cycling enzyme mix.

The chromophoric test reaction was induced with NAD(P) developer measured as absorbance at 450 nm. The chromophoric test reaction was measured as absorbance at 450 nm. The absorbance of the samples was plugged into the equation corresponding to the standard curve, and the NAD(P)total was calculated in the yeast lysate.

The coenzyme content of nine mutant strains (K-1 to K-9) selected in the present invention was measured. The NADtotal of the mutant strains had a minimum of 126 nmole/g and a maximum of 195 nmole/g. The NADtotal of the mutant strain increased at least 7.3 times and up to 10.8 times compared to the type-strain. The NADPtotal content of the mutant strain was a minimum of 2.4 nmole/g and a maximum of 5.8 nmole/g. The NADP total content of the mutant strain increased at least 11.4 times and up to 27.6 times compared to the type-strain.

In the six mutant strains(K-1,4,6,7,8,9), the increase rate of NADPtotal was less than twice the increase rate of NADtotal. The NADPtotal content increase rates of the three novel mutant strains (PicoYP, PicoYP-01, and PicoYP-02) were 25.7, 22.9, and 27.6 times, respectively. The NAD total content increase rates of the three novel mutant strains were 10.8, 9.9, and 11.3 times, respectively. The NADPtotal increase rate of the three novel mutant strains was more than twice the NADtotal increase rate.

### 3-4: Content of Glutathione (GSH)

The glutathione content of the nine mutant strains was measured in the same manner as Example 1-2. The glutathione content of the mutant strains ranged from a minimum of 0.85% to a maximum of 1.05%. The glutathione content of the mutant strain increased at least 2.7 times and up to 3.3 times compared to the type strain. In three novel mutant strains (PicoYP, PicoYP-01, PicoYP-02), the increase rate of ALDH activity and coenzyme content were higher compared to others.

The three novel mutant yeasts (PicoYP, PicoYP-01, PicoYP-02) had similar glutathione production abilities to the existing deposited strains (Kwon P-1, Kwon P-2, Kwon P-3). The three novel mutant yeasts had significantly increased ADH and ALDH enzyme activities and coenzyme contents compared to the existing deposited strains.

TABLE 3

| | Enzyme activity | | Coenzyme concentration | | GSH | | |
|---|---|---|---|---|---|---|---|
| Strain | ADH | ALDH | NADₜₒₜₐₗ | NADPₜₒₜₐₗ | (%) | Name | |
| Type-strain | 74.6 | 2.9 | 17.2 | 0.21 | 0.32 | Reference | KCTC7296 |
| | | | | | | yeast | |
| K-1 | 542.26 | 23.11 | 169.8 | 4.1 | 1.00 | KwonP-1 | KCTC13925BP |
| K-2 | 725.11 | 53.1 | 185 | 5.4 | 0.86 | PicoYP | KCTC14983BP |
| K-3 | 866.41 | 67.4 | 171 | 4.8 | 0.85 | PicoYP-01 | KCTC14984BP |
| K-4 | 625.11 | 31.4 | 176 | 5.1 | 0.98 | KwonP-2 | KCTC14122BP |
| K-5 | 975.29 | 72.16 | 195 | 5.8 | 0.89 | PicoYP-02 | KCTC14985BP |
| K-6 | 458.88 | 16.21 | 154 | 3.1 | 1.05 | — | |
| K-7 | 382.69 | 15.23 | 126 | 2.4 | 1.00 | — | |
| K-8 | 422.17 | 16.19 | 142 | 2.9 | 0.99 | — | |
| K-9 | 533.54 | 20.68 | 167 | 3.2 | 1.00 | KwonP-3 | KCTC14123BP |

TABLE 4

| | Enzyme activity | | Coenzyme concentration | | GSH | | |
|---|---|---|---|---|---|---|---|
| Strain | ADH | ALDH | NADₜₒₜₐₗ | NADPₜₒₜₐₗ | (%) | Name | |
| Type-strain | 1 | 1 | 1 | 1 | 1 | Reference | KCTC7296 |
| | | | | | | yeast | |
| K-1 | 7.3 | 8.0 | 9.9 | 19.5 | 3.1 | KwonP-1 | KCTC13925BP |
| K-2 | 9.7 | 18.3 | 10.8 | 25.7 | 2.7 | PicoYP | KCTC14983BP |
| K-3 | 11.6 | 23.2 | 9.9 | 22.9 | 2.7 | PicoYP-01 | KCTC14984BP |
| K-4 | 8.4 | 10.8 | 10.2 | 17.1 | 3.1 | KwonP-2 | KCTC14122BP |
| K-5 | 13.1 | 24.9 | 11.3 | 27.6 | 2.8 | PicoYP-02 | KCTC14985BP |
| K-6 | 6.2 | 5.6 | 9.0 | 14.8 | 3.3 | — | |
| K-7 | 5.1 | 5.3 | 7.3 | 11.4 | 3.1 | — | |
| K-8 | 5.7 | 5.6 | 8.3 | 13.8 | 3.1 | — | |
| K-9 | 7.2 | 7.1 | 9.7 | 15.2 | 3.1 | KwonP-3 | KCTC14123BP |

### [Example 4] Comparison of Carbon Source Preference

It was investigated the carbon source preference for growth of three mutant strains (KwonP-1, KwonP-2, KwonP-3) with high ALDH and glutathione, for which a domestic patent application was filed on Feb. 18, 2020. Various carbon sources used by the reference yeast strain (KCTC7296) for growth were measured. To find the maximum ability of producing ALDH, it was investigated the carbon source preference for growth of three new mutant strains (Pico YP, Pico YP-01, and PicoYP-02).

The characteristic and novelty of carbon source preference of strains was analyzed by API 50 CHL kit (API systems, BIOMERIEUX, SA, France).

Preparing the 15 ml of conical tube included 8 ml of YPD medium. Each of the seven mutant strains was inoculated into the prepared conical tube.

After culturing the inoculated conical tubes at 30° C. and 200 rpm for 24 hours, each of the seven mutant strains was secured and extracted from the stage of exponential growth phase. To eliminate the influence of the carbon source contained in the residual YPD medium, the yeast was washed three times using a centrifuge. A yeast suspension of 2McFarland concentration was prepared using API 50 CHL medium. The prepared yeast suspension was filled into the tube of the strip. The strip onto which the suspension was dispensed was cultured at 30° C. for 24 hours.

API 50 CHL medium used for API testing was purple. When acids were produced through energy metabolism, API 50 CHL medium turns blue, green, and finally yellow. In the end, it was recorded which type of carbon source was used by mutant strains based on the color change as like: Purple x, Blue +, Green ++, and Yellow +++.

All of the seven mutant strains tested used 19 kinds of carbon sources for energy production and growth: L-arabinose, ribose, D-xylose, D-galactose, D-glucose, D-fructose, D-mannose, mannitol, N-acetyl-glucosamine, arbutin, salicin, cellobiose, maltose, lactose, melibiose, sucrose, trehalose, raffinose, gentiobiose.

Rhamnose was used by only three mutant strains: KwonP-1, PicoYP-01, PicoYP-02. Sorbitol was used by four mutant strains: KwonP-1, KwonP-3, PicoYP-01, PicoYP-02, α-methyl-D-mannoside was used by four mutant strains: type strain, KwonP-1, KwonP-2, PicoYP-02. Amygdalin was used by six mutant strains: KwonP-1, KwonP-2, KwonP-3, PicoYP, PicoYP-01, PicoYP-02. D-turanose was used by four mutant strains: type-strain, KwonP-1, KwonP-3, PicoYP-02. D-tagatose was used by three mutant strains: type-strain, KwonP-3, PicoYP-3. Gluconate was used only by type-strain.

Mannitol and sorbitol, which correspond to alcoholic carbon sources, had a significant effect on yeast growth. The three types of novel mutant strains differed from the other four types of yeast in the type of sugar used for growth. The use of the preferred alcoholic carbon source was slightly different between the three new mutant strains (PicoYP, PicoYP-01 and Pico YP-02) [Table 5].

TABLE 5

| | Reference | Kwon | Kwon | Kwon | Pico | Pico | Pico |
|---|---|---|---|---|---|---|---|
| | yeast | P-1 | P-2 | P-3 | YP | YP-01 | YP-02 |
| L-Arabinose | ++ | +++ | ++ | +++ | ++ | ++ | +++ |
| Ribose | +++ | +++ | +++ | +++ | +++ | +++ | +++ |
| D-Xylose | + | ++ | + | + | ++ | ++ | + |
| D-Galactose | + | +++ | ++ | ++ | +++ | ++ | ++ |
| D-Glucose | ++ | +++ | +++ | ++ | +++ | ++ | ++ |
| D-Fructose | ++ | +++ | ++ | ++ | +++ | ++ | ++ |
| D-Mannose | ++ | +++ | +++ | ++ | +++ | ++ | ++ |
| Rhamnose | | + | | | | ++ | ++ |
| Mannitol | + | + | + | + | ++ | +++ | +++ |
| Sorbitol | | | + | + | | +++ | +++ |
| α-Methyl-D- | +++ | + | + | | | | +++ |
| Mannoside | | | | | | | |
| N-Acetyl- | +++ | +++ | +++ | +++ | +++ | +++ | +++ |
| Glucosamine | | | | | | | |
| Amygdalin | | + | + | + | ++ | ++ | ++ |
| Arbutin | +++ | +++ | +++ | +++ | +++ | +++ | +++ |
| Salicin | +++ | +++ | +++ | +++ | +++ | +++ | +++ |
| Cellobiose | +++ | +++ | +++ | +++ | +++ | +++ | +++ |
| Maltose | ++ | +++ | +++ | +++ | +++ | +++ | +++ |
| Lactose | ++ | ++ | ++ | ++ | ++ | ++ | ++ |
| Melibiose | ++ | + | +++ | +++ | ++ | ++ | +++ |
| Sucrose | ++ | +++ | ++ | ++ | +++ | +++ | ++ |
| Trehalose | ++ | + | ++ | ++ | ++ | ++ | ++ |
| Raffinose | ++ | + | + | ++ | ++ | ++ | +++ |
| Gentiobiose | ++ | ++ | ++ | ++ | ++ | ++ | ++ |
| D-Turanose | + | + | | + | | | + |
| D-Tagatose | ++ | | | + | | | ++ |
| Gluconate | + | | | | | | |

### [Example 5] Changes in ALDH Activity of Mutant Strains in Gastric Juice

When KARC is administered orally, in order for the enzyme activity to be maintained in the intestine, the enzyme activity must be passed safely without being destroyed by stomach acid, which secretes powerful proteolytic enzymes such as pepsin.

NaOH solution was added to artificial gastric fluid at pH=1.17 to artificially generate two simulated solutions at pH=3 and pH=5, which resemble the human gastric environment during food digestion. 1 g of KARC was added to 7 ml of artificial gastric fluid and 7 ml of two simulated solutions and mixed at 36.5° C. for 5, 30, 60, and 90 min respectively. NaOH solution was added to reaction mixture to adjust acidity to pH=7, respectively. 10 ml of sample for analysis were taken from the adjusted solution at pH=7, respectively. The activity of ALDH was analyzed from each sample.

Under the condition of pH=1.17, the ALDH activity of the sample decreased by more than 92.88% compared to the control group during 5 minutes of reaction. Under the condition of pH=1.17, the ALDH activity of the sample decreased by an average of 98.89% for 90 minutes. The ALDH activity of the samples decreased by an average of 96.66% over 90 min at pH=3 and 56.83% at pH=5. Ultimately the ALDH activity at pH=3 and 5 remained relatively higher than that at pH=1.17 during the 90-min reaction.

In detail, the ALDH activity of KwonP-1 (KCTC13925BP) at pH=1.17 decreased by 90.94% compared to the control group to 5.57 unit/g when reacted for 5 minutes. The ALDH activity of KwonP-1 decreased by 98.57% to 0.88unit/g for 90 minutes [FIG. 14]. The enzyme activity at pH=3 and pH=5 remained relatively higher than at pH=1.17. When reacted for 90 minutes, the ALDH activity of KwonP-1 decreased by 96.66% to 5.57 units/g at pH=3, and decreased by 98.57% to 0.88 units/g at pH=5.

The ALDH activity of KwonP-2 (KCTC14122BP) at pH=1.17 decreased by 91.18% to 5.43 unit/g when reacted for 5 minutes. The ALDH activity of KwonP-2 decreased by 98.81% to 0.73unit/g for 90 minutes [FIG. 15]. At pH 3 and pH 5, higher enzyme activity was maintained than at pH 1.17. When reacted for 90 minutes, the ALDH activity decreased by 97.62% to 1.47 units/g at pH=3, and decreased by 56.11% to 26.99 units/g at pH=5.

The ALDH activity of KwonP-3 (KCTC14123BP) at pH=1.17 decreased by 89.99% to 6.16unit/g when reacted for 5 minutes. The ALDH activity of KwonP-3 decreased by 97.85% to 1.32unit/g for 90 minutes [FIG. 16]. At pH 3 and pH 5, higher enzyme activity was maintained than at pH 1.17. When reacted for 90 minutes, the ALDH activity decreased by 92.61% to 4.55 units/g at pH=3, and decreased by 62.31% to 22.18 units/g at pH=5.

The ALDH activity of PicoYP(KCTC14983BP) at pH=1.17 decreased by 92.84% to 4.40unit/g when reacted for 5 minutes. The ALDH activity of Pico YP decreased by 98.33% to 1.03unit/g for 90 minutes [FIG. 17]. Higher enzyme activity was maintained at pH 3 and pH 5. When reacted for 90 minutes, the ALDH activity decreased by 96.66% to 2.05 units/g at pH=3, and decreased by 53.97% to 28.31 units/g at pH=5.

The ALDH activity of PicoYP-01(KCTC14984BP) at pH=1.17 decreased by 95.71% to 2.64unit/g when reacted for 5 minutes. The ALDH activity of PicoYP-01 decreased by 99.76% to 0.15unit/g for 90 minutes [FIG. 18]. At pH 3 and pH 5, higher enzyme activity was maintained than gastric fluid. When reacted for 90 minutes, the ALDH activity decreased by 98.21% to 1.10 units/g at pH=3, and decreased by 58.74% to 25.38 units/g at pH=5.

The ALDH activity of Pico YP-02(KCTC14985BP) at pH=1.17 decreased by 96.66% to 2.05unit/g when reacted for 5 minutes. The ALDH activity of Pico YP-02 decreased by 99.76% to 0.15unit/g for 90 minutes [FIG. 19]. At pH 3 and pH 5, higher enzyme activity was maintained than in gastric juice. When reacted for 90 minutes, the ALDH activity decreased by 98.21% to 1.10 units/g at pH=3, and decreased by 62.08% to 23.32 units/g at pH=5.

pH 1.17 is the pH of the raw gastric juice secreted. When you eat food, the pH rises from 3 to 5 when raw gastric fluids and food mix in the stomach, so it is unlikely that a pH of 1.17 will be reached. Nevertheless, ALDH activity in the mutant strain was retained even at pH 1.17, which is an extreme condition.

In the end, the ALDH enzyme activity of the novel mutant strains (Pico YP, Pico YP-01, PicoYP-02) was maintained at 2 unit/g to 5 unit/g even though it decreased from 92% to 97% under strongly acidic conditions of pH=1.17. 2-5 units of enzyme activity remain, which is sufficient to function in the intestines. It even remained higher at pH=3 and pH=5 compared to pH=1.17. This was the reason for reaching the conclusion that new mutant strains (Pico YP, Pico YP-01, PicoYP-02) could be administered orally.

### [Example 6] Growth Characteristics of 5 L Fermenter Cultures

Each was inoculated into YPD medium (2% peptone, 1% yeast extract, 2% glucose) and primary seed culture was performed at 30° ° C. and 200 rpm for 18 hours. 20 ml of cultured seed was inoculated into 1980 ml of YPD medium and cultured again in 5 L. Cultivation in a 5 L culture tank was carried out at 30° C. and 200 rpm for 48 hours. Growth curve at OD660 nm and enzyme activity were analyzed using 10 ml of sample collected from secondary culture.

The maximum density (OD660 nm) of KwonP-1 (KCTC13925BP) was 134.4. The maximum density of KwonP-1 was 4.35% higher than that of the type-strain (KCTC7296). The growth curve characteristics and specific growth rate (OD660 nm/hr) of KwonP-1 were similar to those of the type-strain. The ALDH activity of KwonP-1 was 33.6 unit/g. The ALDH activity of KwonP-1 was 11.96 times higher than that of the type-strain [FIG. 20].

The maximum density (OD660 nm) of KwonP-2 (KCTC14122BP) was 133.8. The maximum density of KwonP-2 was 3.88% higher than that of the type-strain. The growth of KwonP-2 ended earlier than that of the type-strain. The specific growth rate (OD660 nm/hr) of KwonP-2 was 14.8% higher than that of the type-strain. The ALDH activity of KwonP-2 was 31.5 unit/g. The ALDH activity of KwonP-2 was 11.21 times higher than that of the type-strain [FIG. 21].

The maximum density (OD660 nm) of KwonP-3 (KCTC14123BP) was 134.1. The maximum density of KwonP-3 was 4.12% higher than that of the type-strain. The growth of KwonP-3 ended earlier than that of the type-strain. The specific growth rate (OD660 nm/hr) of KwonP-3 was 6.08% higher than that of the type-strain. The ALDH activity of KwonP-3 was 29.5 unit/g. The ALDH activity of KwonP-3 was 10.5 times higher than that of the type-strain [FIG. 22].

The maximum density (OD660 nm) of PicoYP (KCTC14983BP) was 123.8. The maximum density of PicoYP was 3.88% higher than that of type-strain. The growth curve characteristics of PicoYP were similar to those of type-strain. The specific growth rate (OD660 nm/hr) of PicoYP was 6.22% higher than that of the type-strain. The ALDH activity of Pico YP was 44.2 unit/g. The ALDH activity of PicoYP was 15.73 times higher than that of the type-strain [FIG. 23].

The maximum density (OD660 nm) of PicoYP-01 (KCTC14984BP) was 126.9. The maximum density of PicoYP-01 was 1.47% higher than that of the type-strain. The growth curve characteristics of PicoYP-01 were similar to those of type-strain. The specific growth rate (OD660 nm/hr) of PicoYP-01 was 2.14% higher than that of the type-strain. The ALDH activity of PicoYP-01 was 47.1 unit/g. The ALDH activity of PicoYP-01 was 16.76 times higher than that of the type-strain [FIG. 24].

The maximum density (OD660 nm) of PicoYP-02 (KCTC14985BP) was 148.1. The maximum density of PicoYP-02 was 14.99% higher than that of the type-strain. The growth curve of PicoYP-02 was located at the top compared to the type-strain. The specific growth rate (OD660 nm/hr) of PicoYP-02 was 9.64% lower than that of the type-strain. The ALDH activity of PicoYP-02 was 52.68 unit/g. The ALDH activity of PicoYP-02 was 18.75 times higher than that of the type-strain [FIG. 25].

### [Example 7] Preparation of Mutant Strain Lysates (KARC)

To preserve the enzymes (ALDH, ADH) contained in the mutant enzyme lysate, proteases were removed and inhibited. To preserve the enzymes (ALDH, ADH) contained in the mutant enzyme lysate cell debris was removed. The dried product or lysate of the mutant strain was mixed to prepare the KARC composition.

The mutant strain and the medium in which it was cultured contained various substances, such as yeast metabolites and proteolytic enzymes secreted by yeast. In order to extract and preserve ALDH, coenzyme, and glutathione present in yeast, it is necessary to sufficiently remove substances outside the yeast fungus, and for this purpose, a washing process was performed. Washing of the mutant strain was carried out by dispensing 40 ml of culture medium into 50 ml conical tubes, centrifuging at 13,000 rpm for 15 minutes, and removing the supernatant.

As a result of centrifugation, residual medium remained inside the pellet produced by the yeast bacteria clumping together. After adding 30 ml of purified water, the pellet was sufficiently loosened by vortex, and the previous process was repeated three times to sufficiently remove the remaining medium.

The ethanol resistance of yeast is known to be up to 13%, and yeast bacteria die when exposed to high concentrations of ethanol. The washed pellet was sufficiently dissolved using 10 ml of 20% ethanol solution to induce the death of yeast bacteria. The pellet dissolved in ethanol was stirred at 100 rpm for 30 minutes to proceed with the yeast death process. When the reaction time was completed, 30 ml purified water was added to lower the ethanol concentration to 5%. The previous washing process was repeated three times to sufficiently remove ethanol.

To preserve ALDH and ADH from the decomposition action of proteases present in yeast cells, 10 ml of 1×PBS was prepared by dissolving 2 tablets of protease inhibitor (Pierce protease inhibitor mini tablets, EDTA-free, Thermo Scientific). The above solution was added to the washed yeast pellet and sufficiently released.

To prepare a lysate of the mutant strain prepared in the present invention, 4 g of glass beads were added and stirred to break the yeast cell wall. To prevent denaturation of the enzyme due to the heat generated during the process of crushing the yeast, vortex for 30 seconds and ice incubation for 30 seconds were repeated six times.

After the yeast cell wall disruption was completed, 10 ml of 100 mM potassium phosphate buffer was added and mixed by vortex for 3-5 seconds. It was centrifuged at 13,000 rpm for 15 minutes to remove cell structures such as yeast cell walls and glass beads. The supernatant was filtered through a 0.2 μm filter (Minisart® Syringe Filter, Sartorius, Goettingen, ermany) to prepare the KARC composition.

To preserve the enzymes (ALDH, ADH) contained in the mutant enzyme lysate, intracellular proteases were removed and inhibited, and cell debris such as cell walls were removed. The KARC composition was prepared with a lysate selected from the 6 mutant strains (KwonP-1, KwonP-2, KwonP-3, Pico YP, PicoYP-01, PicoYP-02), or a mixture thereof in a free ratio [Table 6].

KARC 1 was manufactured from KwonP-1. The enzyme activity of ADH and ALDH of KARC 1 were 461.4 unit/g and 28.6unit/g, respectively. In KARC 1, the content of coenzymes of NADtotal and NADPtotal were 176.2 nmole/g and 5.1 nmole/g, respectively. The GSH content of KARC 1 was 0.98 wt %.

KARC 2 was manufactured from KwonP-2. the enzyme activity of ADH and ALDH of KARC 2 were 482.1 unit/g and 29.8unit/g, respectively. In KARC 2, the content of coenzymes of NADtotal and NADPtotal were 175.4 nmole/g and 5.2 nmole/g, respectively. The GSH content of KARC 2 was 0.96 wt %.

KARC 3 was manufactured from KwonP-3. the enzyme activity of ADH and ALDH of KARC 2 were 477.5 unit/g and 28.1 unit/g, respectively. In KARC 3, the content of coenzymes of NADtotal and NADPtotal were 177.2 nmole/g and 5.1 nmole/g, respectively. The GSH content of KARC 3 was 1.00 wt %.

KARC 4 was manufactured from PicoYP. the enzyme activity of ADH and ALDH of KARC 2 were 586.8 unit/g and 33.8 unit/g, respectively. In KARC 4, the content of coenzymes of NADtotal and NADPtotal were 184.3 nmole/g and 5.7 nmole/g, respectively. The GSH content of KARC 4 was 0.84 wt %.

KARC 5 was manufactured from PicoYP-01. the enzyme activity of ADH and ALDH of KARC 5 were 621,6 unit/g and 38.2 unit/g, respectively. In KARC 5, the content of coenzymes of NADtotal and NADPtotal were 186.9 nmole/g and 5.6 nmole/g, respectively. The GSH content of KARC 5 was 0.84 wt %.

KARC 6 was manufactured from PicoYP-02. the enzyme activity of ADH and ALDH of KARC 5 were 664,1 unit/g and 41.6 unit/g, respectively. In KARC 6, the content of coenzymes of NADtotal and NADPtotal were 195.0 nmole/g and 5.8 nmole/g, respectively. The GSH content of KARC 6 was 0.88 wt %.

KARC was manufactured by freely mixing dry powders and lysates prepared from six deposit strains. The average enzyme activities of ADH and ALDH in the composition of KARC were 547.6 unit/g and 33.1 unit/g, respectively. The average contents of coenzyme NADtotal and coenzyme NADPtotal in the composition of KARC were 180.4 nmole/g and 5.4 nmole/g, respectively. The average content of glutathione in the composition of KARC was 0.84 wt %.

The aldehyde decomposition ability of KARC was kept on during the lysate production process. KARC showed the ability to remove endogenous aldehydes such as HNE, MDA, and 3,4-dihydroxyphenyl acetaldehyde (DOPAL).

TABLE 6

| | | ADH | ALDH | NADtotal | NADPtotal | GSH |
|---|---|---|---|---|---|---|
| Name | Strain | (Unit/g) | (Unit/g) | (nmole/g) | (nmole/g) | (wt %) |
| KARC1 | KwonP-1 | 461.4 | 28.6 | 176.2 | 5.1 | 0.98 |
| KARC2 | KwonP-2 | 482.1 | 29.8 | 175.4 | 5.2 | 0.96 |
| KARC3 | KwonP-3 | 477.5 | 28.1 | 177.2 | 5.1 | 1.00 |
| KARC4 | PicoYP | 586.8 | 33.8 | 184.3 | 5.7 | 0.84 |
| KARC5 | PicoYP-01 | 621.6 | 38.2 | 186.9 | 5.6 | 0.83 |
| KARC6 | PicoYP-02 | 664.1 | 41.6 | 195.0 | 5.8 | 0.88 |
| KARC | average | 547.6 | 33.1 | 180.4 | 5.4 | 0.91 |

[Example 8] Analysis of sequence of ALDH contained in the mutant strain. It was investigated the differences between both ALD (yeast aldehyde dehydrogenase) of the mutant strains and parent strain. Whole genome sequencing was performed on the parent strain and mutant strains of KwonP-1, KwonP-2, KwonP-3, PicoYP, PicoYP-01, and PicoYP-02. The mutant strain cells were obtained by culturing pure strains on solid medium. The genome sequence of the mutant strain obtained were analyzed.

Among ALDs (yeast aldehyde dehydrogenases) in the novel mutant strains, ALD2(SEQ ID NO:3) was found to be condensed with ALD3(SEQ ID NO:4) on chromosome 13. A non-coding region of 689 nucleotides was located between the ALD2 and ALD3 coding genes.

The ALD2 and ALD3 existed continuously in the same genome. ALD2 and ALD3 encoded respective aldehyde dehydrogenases. ALD2 coding gene was almost similar to ALD3, consist of 1,521 nucleotides and 506 amino acids, but had an 8.2% difference in sequence. ALD2 and ALD3 they were identified as separate aldehyde dehydrogenases that differed from each other in 125 base sequences (8.2%).

In the six mutant strains (KwonP-1, KwonP-2, KwonP-3, PicoYP, PicoYP-01, PicoYP-02), there is no stop codon at the end of the ALD2 sequence, so proteins are synthesized continuously. As a result, a new, larger ALDH enzyme is created by linking a part of ALD2 and ALD3[SEQ ID NO: 1].

ALD2[SEQ ID NO. 3] of the type-strain (KCTC7296) consisted of 30 nucleotide sequences (5′-GTTCACATAAATCTCTCTTTGGACAACTAA-3′) coding 9 amino acids (N-VHINLSLDN-C) at the terminal, excluding the stop codon.

ALD2 of the six mutant strains consisted of specific 42 nucleotide sequences (5′-AGATATAGATTATACACATTTAGAAAATTAGCCAAAAGAAAA-3′) coding 14 amino acids (N-RYRLYTFRKLAKRK-C) between 5′-terminal of ALD2 and ALD3, [SEQ ID NO. 2].

There was no stop codon at the end of the sequence in ALD2 coding gene by deleted from the 1492^{nd} nucleotide of ALD2 to 647^{th} nucleotide of non-coding region. Finally, the six deposited mutant strains had new mutated gene consist of total 3,054 bases coding novel ALD. [SEQ ID NO: 1].

### [Example 9] Observation of In Vitro Decompostion of Succinic Semialdehydes (SSA) by KARC

The present invention confirmed the effect of KARC in reducing succinic semialdehydes (SSA).

### 9-1: Reaction of Succinic Semialdehydes (SSA)

Potassium chloride (KCl) was dissolved in a 50 mM of pH 7.5 HEPES buffer solution to be 200 Mm for buffer.

For experiments with SSA, 845 μl of the buffer, 15 μl of 100 mM EDTA aqueous solution, 30 μl of 100 mM NADP+ aqueous solution, 10 μl of 10 mM SSA in acetonitrile solution, and 10 μl of 300 mg/mL KARC were dispensed into microtubes. As a negative control, 845 μl of buffer solution, 15 μl of 100 mM EDTA aqueous solution, 30 μl of 100 mM NADP+ aqueous solution, 100 μl of 10 mM SSA in acetonitrile solution, and 10 μl of DW were dispensed into a microtube. The reactants were shakes at 30° C. or 37° C. for 1 hour or 3 hours using thermoshaker.

### 9-2: Pre-Processing Before HPLC Analysis

For experiments using the representative aliphatic aldehydes: SSA, acetaldehyde, a 500 μl of each reaction was aliquoted into a microtube at the end of the reaction. 470 μl of methanol, 20 μl of 50 mM DNPH in acetonitrile solution, and 10 μl of 6N HCl were additionally dispensed into the microtube containing the reaction solution, and heated at 70° C. for 40 minutes. Alternatively, 480 μl of methanol, 10 μl of 100 mM DHBA in acetonitrile solution, and 10 μl of 6N HCl were added and heated at 70° ° C. for 40 min. After the heated solution was cooled, 10 μl was quantified and injected into HPLC for analysis.

### 9-3: HPLC Analysis

HPLC system (Waters Alliance 2690/2695 HPLC with Waters 2996 PDA detector) was used for analysis. The analytical column was 150 mm×4.6 mm i.d. packed with C18, 5 μm particle size (Shimadzu Scientific Instruments, Kyoto, Japan).

In gradient, it started at 80% of water (1v/v % trifluoroacetic acid) and deployed in reverse phase to 20% after 15 minutes. Absorbance was analyzed at wavelengths of 254 nm, 310 nm, or 360 nm with an ultraviolet detector.

The results were confirmed by the progress of the reaction in which aldehyde was consumed through the reduction of DNPH-aldehyde conjugates or DHBA-aldehyde conjugates in the experimental group compared to the negative control group. [FIGS. 21, 22, 23].

### [Example 10] In Vivo Acetaldehyde (Ach) and Malondialdehyde (MDA) Reduction Effect by Oral Administration of KARC

For the acetaldehyde and MDA animal experiments, 5-week-old male Sprague Dawley (SD) rats (Rat) were used. The KARC composition was orally administered to rats at 10 units/kg or 20 units/kg, and alcohol (3 g/kg) was orally administered to the rats 30 minutes after KARC injection.

After the administration was completed, blood samples were collected from the tail vein at 0, 1, 3, 5, and 8 hours after KARC injection, and after centrifugation, plasma was stored at −80° C. [FIGS. 7, 8].

7-week-old male Wistar rats (7 weeks old, 250 g, n=8-10) were used. Rotenone solution (2.5 mg rotenone/ml, 20 μl DMSO/ml) was prepared using natural oil (middle chain triglycerides). Mice were intraperitoneal injection administered rotenone solution (2.5 mg/kg) daily for 60 days.

Two administration methods were employed to confirm the Parkinson's disease prevention and treatment effects of KARC. KARC (20 units/kg) was administered orally at the same time as rotenone administration to observe the effect of preventing Parkinson's disease. KARC (20 units/kg) or L-dopa were administered orally at the two weeks after rotenone administration to observe the effect of therapeutic Parkinson's disease. To quantify dopamine, brain tissues were isolated and stored at −80° ° C. in liquid nitrogen. [FIG. 9, 10].

### 10-1: Acetaldehyde Reduction Effect by Oral Administration of KARC

The total acetaldehyde reduction effect by oral administration of KARC was assessed using an Acetaldehyde assay kit (LSBio, Seattle, WA, USA). 20 μl of each sample was dispensed into two wells of a 96 well plate. 80 μl of working reagent (75 μl assay buffer, 8 μl NAD/MTT, 1 μl Enzyme A, 1 μl Enzyme B) was dispensed into one well. In the remaining well, 80 μl of blank working reagent (75 μl assay buffer, 8 μl NAD/MTT, 1 μl Enzyme B) was dispensed. The plate after dispensing was lightly mixed and reacted at room temperature for 30 minutes. When the reaction was completed, the absorbance was measured at 565 nm (520-600 nm).

The concentration of acetaldehyde reached the maximum 1 hour after ethanol administration and showed a tendency to decrease in the KARC composition administration group. In the KARC administration group, acetaldehyde concentration significantly decreased compared to the control group (Vehicle) 1, 3, and 5 hours after ethanol administration. In the KARC high-dose administration group (F), the blood acetaldehyde concentration was 0.356, 0.224, and 0.091 mM, respectively, which decreased by 39.2%, 58.4%, and 72.1% compared to the control group [FIG. 7].

### 10-2: MDA Reduction Effect by Oral Administration of KARC

Total malondialdehyde content in blood was analyzed using the OxiTec™ TBARS assay kit according to the manufacturer's protocol (ZeptoMetric, Buffalo, NY, USA). 100 μl sample, 100 μl 8.1% SDS solution, and 4 ml color indicator (TBA, 10% NaOH solution, 20% acetic acid) were added to the conical tube, and then reacted in a constant temperature water bath at 95° C. for 60 minutes. After completion of the reaction, the sample was centrifuged at 4° C. and 1,600 rpm for 10 minutes and stabilized at room temperature for 30 minutes. 150 μl of supernatant was transferred to a 96 well plate, and absorbance was measured at 530-540 nm.

In the control group (Vehicle), the concentration of MDA in the blood reached the maximum 3 hours after ethanol administration, whereas in the group administered KARC, it reached the maximum value 1 hour after ethanol administration. The concentration of MDA in the blood decreased, showing a significant difference from the control group 3 and 5 hours after ethanol administration. The blood MDA concentration of the KARC high-dose administration group (F) was 0.232 and 0.137 μM, respectively, a decrease of 80.4% and 86.3% compared to the control group [FIG. 8].

These results showed that oral administration of KARC was effective in reducing various endogenous aldehydes such as acetaldehyde and malondialdehyde in the blood.

### [Example 11] Effect of Reducing Oxidative Stress

Reactive oxygen species or oxidative stress increases when drinking alcohol due to excessive acetaldehyde (Ach) produced by alcohol dehydrogenase (ADH). Aldehyde dehydrogenase (ALDH) acts to convert it into acetic acid and excrete it out of the body. In the case of aldehyde dehydrogenase gene mutation or excessive aldehyde caused by excessive alcohol cause peroxidation of fat.

The resulting acetaldehyde and malondialdehyde worsen oxidative stress and interfere with mitochondrial energy metabolism. Endoplasmic reticulum stress is induced through the accumulation of denatured proteins in cells, leading to cell death.

The concentration of blood acetaldehyde was measured over time following alcohol consumption [FIG. 11]. The area under the curve (AUC) of blood acetaldehyde (Ach) was 13.02±1.18 mg·h/dL for alcohol consumption alone. When administered at a dose of KARC 10 units/kg, the area under the curve (AUC) of blood acetaldehyde (Ach) significantly decreased by 26.13% compared to alcohol consumption alone, measuring 9.39±1.07 mg·h/dL (P=0.005).

At a dose of KARC 20 units/kg administration, the AUC of blood acetaldehyde (Ach) decreased significantly by 55.71% compared to alcohol consumption alone, measuring 5.22±0.99 mg·h/dL (P<0.001). When comparing the KARC 10 units/kg administration group with the KARC 20 units/kg administration group, the blood acetaldehyde (Ach) in the KARC 20 units/kg group decreased significantly (P=0.034). KARC demonstrated dose-dependent reduction in the total amount of blood acetaldehyde (Ach) over time.

The reduction in blood acetaldehyde (Ach) concentration due to KARC administration has a positive impact on reducing oxidative stress and promoting health.

The concentration of blood malondialdehyde (MDA) was measured during the chemotherapy period [FIG. 12]. The concentration of blood MDA in the control group was 0.607±0.161 μM. The group undergoing treatment with KARC showed a significant 63.3% reduction in blood MDA concentration, measuring 0.223±0.033 μM compared to the control group (P<0.001).

In the control group, the blood MDA concentration ranged from 0.427 μM to 0.885 μM with a substantial variability. In the KARC administration group, the range was significantly reduced, with values ranging from 0.158 μM to 0.269 μM. This not only confirmed the effect of reducing blood MDA concentration but also stabilizing it, as demonstrated in [FIG. 13].

Various factors, such as drug intake, stress, and intense physical exercise, lead to an increase in intracellular reactive oxygen species. This triggers lipid peroxidation reactions and oxidative processes in endogenous amines such as dopamine, norepinephrine, serotonin, histamine, and more. Reactive aldehyde compounds, including 4-hydroxynonenal (HNE), malondialdehyde (MDA), acetaldehyde (Ach), and dopamine-induced aldehyde, accumulate within cells, exacerbating oxidative stress.

These aldehydes subsequently react with surrounding proteins and undergo secondary metabolic processes to form stable end products such as Malondialdehyde-acetaldehyde adduct (MAA) and Malondialdehyde lysine adducts (M-lys adducts), known as Advanced Lipid Peroxidation End Products. The accumulation of these products exerts toxic effects on various cells, further intensifying oxidative stress.

This cumulative oxidative stress disrupts mitochondrial energy metabolism within cells and leads to the buildup of aldehyde intermediates in aldehyde-based sugar metabolism, including methylglyoxal (MG) and glyceraldehyde-3-phosphate (GA3P). The chain reaction involving aldehydes results in the accumulation of stable final glycoxidation products known as advanced glycation end products (AGEs), which weaken intracellular antioxidant defense systems like glutathione (GSH). These processes elevate endoplasmic reticulum (ER) stress, leading to increased cellular apoptosis in nerve cells.

The increase in reactive oxygen species and oxidative stress is associated with elevated levels of reactive aldehydes like HNE and MDA, as well as modified proteins such as advanced glycation end products (AGEs) and advanced lipid peroxidation end products (ALEs). This cascade of events is known to involve mutual reinforcement and amplification, leading to heightened endoplasmic reticulum stress (ER stress).

KARC administration effectively regulated malondialdehyde, a marker for active oxygen and oxidative stress, demonstrating the potential for reducing oxidative stress and improving the constancy of endoplasmic reticulum (ER) stress. KARC significantly reduced malondialdehyde concentrations in the bloodstream, illustrating its capability to reduce active oxygen and oxidative stress. By lowering the levels of acetaldehyde and malondialdehyde in human blood, KARC exhibited its potential to prevent and remedy ER stress through the reduction of active oxygen and oxidative stress. This suggests that by modulating intracellular active oxygen and oxidative stress, KARC inhibits neuronal cell apoptosis, consequently suppressing and preventing Parkinson's disease. This leads to improvements in behavioral and motor functions.

### [Example 12] Acute Oral Administration Test

### 12-1. Preparation of Experimental Animals

The experimental animals were female and male ICR mice (7 weeks old). The received ICR mice were acclimatized for 7 days. The general symptoms of the adopted mice were observed during the acclimatization period, and only healthy animals were used for short-term administration toxicity tests. Feed and water were consumed ad libitum. Based on the average body weight of about 20 g the day before oral administration, groups were separated into 10 groups, 5 for each group, and 5 for each group.

### 12-2. Administration of Test Substances

The test substance was prepared by dissolving it in physiological saline so that the dosage for experimental animals was 0, 750, 3,000, and 5,000 mg/kg, respectively, based on the content of the mutant yeast lysate KARC of the present invention.

The standards for administered dosage were in accordance with the Ministry of Food and Drug Safety's Korea national Toxicology Program (KNTP) toxicity test manual. The maximum application dose of 5,000 mg/kg guided by the KNTP manual was set as the maximum concentration for this experiment. The samples prepared for each group were orally administered once to each test animal. For the normal group (G1), physiological saline was administered.

### 12-3. Observation and Autopsies

For animals in all test groups, symptoms of mice were observed at least once a day from the date of acquisition to the date of necropsy. Symptoms were observed for 7 days after oral administration. After observing the rat's symptoms, an autopsy was performed. During the autopsy of the rat, changes in each organ were observed with the naked eye.

A single-dose toxicity test of the ALDH-containing KARC composition of the present invention was conducted using mice. As a result, no cases of mouse death are observed for 7 days at concentrations of the mutant yeast KARC up to 5,000 mg/kg. No unusual features, such as weight gain or changes in feed intake, were found in the mice. No unusual findings were found in the autopsy results conducted after the end of observation

### [Example 13] Preparation of Food and Pharmaceutical Compositions for Alleviating Tremor and Oxidative Stress by Decomposing Endogenous Alcohol and Aldehydes

Food and pharmaceutical compositions containing KARC as an active ingredient for alleviating tremor and oxidative stress were prepared. It is possible to prepare food or pharmaceutical compositions of various composition ratios containing KARC powder. As an example, the powder composition according to the present invention has the function of suppressing tremor and oxidative stress through ingestion of 13 g of the composition twice a day. The weight ratio between the components and phases of the food or pharmaceutical composition containing the powder composition is shown in [Table 7].

TABLE 8

| | Ingredient | Ratio (wt %) |
|---|---|---|
| Food and Drug Composition | KARC dry powder | 50 |
| for Suppressing Tremor of | Fructo-oligosaccharides | 9 |
| Movement Disorder | Stevia | 5 |
| | Citric acid anhydrous | 10 |
| | Iso-malto | 4.3 |
| | Xylitol | 2.5 |
| | Citrus juice Powder | 6.2 |
| | Citrus Flavors Powder | 13 |

### Industrial Applicability

In the food and pharmaceutical composition, KARC dry powder, excipients, and natural sweeteners such as fructo-oligosaccharides, enzyme-treated stevia (Stevia), anhydrous citric acid, iso-maltodextrins (Iso-malto), and xylitol, citrus juice powder, and citrus flavor powder were added. Processing and testing of raw materials and final products of food or pharmaceutical compositions were conducted in accordance with the general test methods and the Health Functional Foods Act described in the Korean Food Code.

KARC-containing food or pharmaceutical compositions decompose endogenous aldehydes and exhibit the effect of Suppressing Tremor or Movement Disorder.

Through the above examples, the mutant yeast composition KARC containing aldehyde dehydrogenase was described in detail: manufacturing methods, pharmacological effects, administration methods, therapeutically effective doses for disease models, short-term administration acute toxicity, and representative examples of food or pharmaceutical compositions. Although the efficacy of KARC has been described in detail through the above examples, these are only examples of the present invention.

A person skilled in the art can easily derive various modifications and other embodiments equivalent to the present invention from the above-described embodiments of the present invention.

Even foods or therapeutic agents containing a modified form of aldehyde dehydrogenase that embodies the technical gist of the present invention described in the patent claims fall within the scope of legal protection of the present invention.

1. A food composition that suppresses or improves symptoms of tremor or movement disorder, containing an aldehyde dehydrogenase encoded by a gene with more than 98% homology to the gene of SEQ ID NO: 1.

2. The food composition for suppressing tremor or movement disorder of claim 1, comprising an aldehyde dehydrogenase encoded by the gene of SEQ ID NO: 1 including SEQ ID NO: 2.

3. The food composition for suppressing tremor or movement disorder according to claim 2, wherein the aldehyde is an endogenous aldehyde.

4. The food composition for suppressing tremor or movement disorder of claim 3, wherein the endogenous aldehyde is an endogenous aldehyde produced by oxidation of alcohol or an endogenous amine compound.

5. The food composition for suppressing tremor or movement disorder of claim 3, wherein the endogenous aldehyde is selected from the group consisting of acetaldehyde, malondialdehyde (MDA), glutamate semialdehyde (GSA) and succinic semialdehyde (SSA).

6. The food composition for suppressing tremor or movement disorder according to claims 2, wherein the aldehyde dehydrogenase in contained in any one or a mixture thereof selected from the group consisting of Saccharomyces cerevisiae KCTC13925BP, KCTC14122BP, KCTC14123BP, KCTC14983BP, KCTC14984BP, and KCTC14985BP.

7. A food composition for suppressing tremor or movement disorder, containing any one or a mixture thereof selected from the group consisting of Saccharomyces cerevisiae KCTC13925BP, KCTC14122BP, KCTC14123BP, KCTC14983BP, KCTC14984BP, and KCTC14985BP.

8. A pharmaceutical composition for suppressing tremor or movement disorder, containing an aldehyde dehydrogenase encoded by a gene with more than 98% homology to the gene of SEQ ID NO: 1.

9. The pharmaceutical composition for suppressing tremor or movement disorder of claim 8, further comprising an aldehyde dehydrogenase encoded by the gene of SEQ ID NO: 1 including SEQ ID NO: 2.

10. The pharmaceutical composition for suppressing tremor or movement disorder of claim 9, wherein the aldehyde dehydrogenase is contained in any one or a mixture thereof selected from the group consisting of Saccharomyces cerevisiae KCTC13925BP, KCTC14122BP, KCTC14123BP, KCTC14983BP, KCTC14984BP, and KCTC14985BP.

11. A pharmaceutical composition for suppressing tremor or movement disorder, comprising a lysate of any one or a mixture thereof selected from the group consisting of Saccharomyces cerevisiae KCTC13925BP, KCTC14122BP, KCTC14123BP, KCTC14983BP, KCTC14984BP, and KCTC14985BP.
